(19) United States
(12) Patent Application Publication
(10) Pub. No.: US 2004/0005351 A1
(43) Pub. Date: Jan. 8, 2004

(54) Polymeric micelle formulations of hydrophobic compounds and methods

(76) Inventors: Glen S. Kwon, Waunakee, WI (US); Devalina Law, Libertyville, IL (US); Monica Adams, East Brunswick, NJ (US); Karen E. Kostick, Madison, WI (US); Eric A. Schmitt, Libertyville, IL (US)

Correspondence Address: GREENLEE WINNER AND SULLIVAN P C, 5370 MANHATTAN CIRCLE, SUITE 201, BOULDER, CO, 80303, US

(21) Appl. No.: 10/404,926

(22) Filed: Mar. 31, 2003

Related U.S. Application Data
(60) Provisional application No. 60/368,771, filed on Mar. 29, 2002.

(51) Int. Cl. B01J 13/04; A61K 9/127; A61K 9/14; A61K 31/715; A61K 31/7012

(52) U.S. Cl. 424/450; 424/486; 514/53; 514/61; 264/4.1

(57) ABSTRACT

Provided are cosolvent evaporation methods and compositions for improving the solubility of hydrophobic compounds, including therapeutic agents such as anticancer drugs, polyene antibiotics, antilipidemic agents, and hydrophobic compounds used in various industries, and/or for reducing the toxicity of certain hydrophobic therapeutic agents, especially polyene antibiotics, in particular, Amphotericin B (AmB), and therapeutics such as paclitaxel, tamoxifen, an acylated prodrug or an acylated cis-platin, by incorporating these agents within micelles comprising an amphiphilic block-forming copolymer.

### CROSS REFERENCE TO RELATED APPLICATION

This application claims benefit of United States Provisional Application 60/368,771, filed Mar. 29, 2002, which is incorporated by reference herein.

### ACKNOWLEDGMENT OF FEDERAL RESEARCH SUPPORT

This invention was made, at least in part, with funding from the National Institutes of Health (NIH grant AI-43346). Accordingly, the United States Government has certain rights in this invention.

### BACKGROUND OF THE INVENTION

The field of the present invention is the area of methods of formulating hydrophobic compounds for use in aqueous systems, especially pharmaceutical compositions for medical and/or veterinary use, in particular, methods of formulating relatively insoluble and/or toxic materials such as antifungal agents, e.g., amphotericin B (AmB) and nystatin, and anticancer agents, e.g., paclitaxel, so that solubility in aqueous milieus is improved and so that toxicity is reduced, release is controlled and in at least some instances, the stability of the formulation is improved. For example, the polyene antibiotic amphotericin B is encapsulated in poly(ethylene glycol)-block-poly(L-amino acid) (PEG-b-PLAA) micelles or in poly(ethylene glycol)-block-poly(ester) micelles. Other targets of the present formulation technology include other hydrophobic compounds such as flavoring agents, pigments, agricultural chemicals, fungicides and insecticides.

Currently, potentially therapeutic molecules have properties that can result in their classification as “challenging” (poorly water soluble) compounds. Such molecules have favorable in vitro capabilities, however due to characteristics such as poor aqueous solubility, toxicity, chemical instability, and limited cellular permeability, these compounds require formulation to be therapeutically effective (Davis, S. S. et al. (1998) Int. J. Pharm. 179, 2). Micelle systems based on amphiphilic block copolymers (ABCs) have been used to formulate such challenging drugs (Jones, M. C et al. (1999) Eur. J. Pharm. Biopharm. 48, 101). ABCs, comprised of a hydrophobic and hydrophilic block, can assemble into a microphase separated, core/shell architecture in a selective solvent. In an aqueous environment, the hydrophobic drug can be encapsulated into the hydrophobic core of the ABC micelle and have aqueous solubility provided by a poly(ethylene glycol), PEG, corona (shell). Due to their nanoscopic dimensions and stealth properties imparted by a PEG corona, micelles may have long-term circulation capabilities. During the circulation period, the ABC may gradually release drug and eventually dissociate and be eliminated from circulation.

In human medicine, fungal infections are, in part, associated with immune-compromised patients such as those infected with HIV, patients who have been subjected to anticancer therapeutics or immune suppressive drugs after organ transplants, and the elderly. Fungal infections fall into two categories: systemic (deep) mycoses and superficial mycoses which involve the skin or mucous membranes. The dermatophytic fungi infect the skin, hair and nails; etiological agents include Epidermiphyton spp., Trichophyton spp. and Microspermum spp. Generally, infections of the mucous membranes are due to infections with Candida albicans. The systemic mycoses are serious and often life-threatening. They include cryptococcosis, systemic candidiasis, aspergillosis, blastomycosis, histoplasmosis, coccidiodomycosis, paracoccidioidomycosis, phycomycosis, torulopsosis, among others.

The three families of drugs used to treat fungal infections are the polyenes, imidazoles and antimetabolites. The polyenes include nystatin, which is generally used for superficial infections only, and amphotericin B. Mepartricin and natrimycin are other polyenes with antifungal activities.

Ketoconazole, itraconazole, miconazole and thiabendazole are imidazoles with antifungal activity. They act by inhibiting cytochrome activity and by interfering with ergosterol synthesis. Flucytosine is an antimetabolite which has been used in the treatment of systemic mycoses. It is converted in vivo to 5-fluorouracil, which inhibits thymidylate synthetase.

Amphotericin B (AmB) has an affinity for membranes with a relatively high ergosterol content; it forms transmembrane channels which allow the passage of potassium and other small molecules. Because the AmB is very toxic, especially in aggregates, and has numerous side effects, it must be given in a hospital setting, adding to treatment costs. There is some evidence (Beringue et al. (1999) J. Gen. Virol. 80, 1873-1877; Beringue et al. (2000) J. Virol. 74, 5432-5440) that certain polyenes may inhibit the progression of scrapie infections.

Despite its low solubility in water and the toxicity problems, AmB is one of the drugs of choice for treating fungal infections. Notably, the development of resistance to AmB is very rare. Numerous strategies have been employed to improve its solubility in aqueous systems and to reduce its toxicity. Strategies for the improvement of solubility and toxicity have included formulation with surfactant, e.g. deoxycholate, liposome encapsulation, encapsulation in polyethylene glycol-complexed liposomes and encapsulation with various amphiphilic polymeric materials.

The potential of nanoparticles (made up of ABCs) that mimic the size of natural carriers such as viruses and lipoproteins as drug delivery vehicles has gained much attention recently. The small size (about 40 nm) of the ABC micelles is compelling for challenging molecules owing to the relative safety after injection, the ability to distribute to sites of disease or neoplasia and ultimately engineering for site-specific drug delivery.

Due to the properties of the covalently joined blocks, ABCs can assemble into a microphase-separated structure in selective solvents. If the solvent is water, the hydrophobic blocks organize to form the core region due to hydrophobic interactions. The hydrophilic blocks then form the shell, or corona, shielding the hydrophobic core regions from the aqueous environment. The hydrophobic core serves as a depot for challenging (poorly water soluble) compounds, which partition into the core during the micelle assembly process. Through control of micelle assembly, poorly water soluble drugs have been loaded into the diblock copolymer micelles. It is after the inception of micellization that the drug is encapsulated by the ABC micelles. Micellization onset parameters such as the critical micelle concentration (cmc) and critical water content (cwc) are established to determine the point at which drug encapsulation begins. ABC micelles have lower cmcs than low molecular weight surfactants, which results in increased thermodynamic stability.

The choice of the hydrophilic block polyethylene glycol (PEG) imparts additional benefits. PEG is nontoxic, highly flexible and highly hydrated. The high surface density and long length of the PEG chains prevents opsonization and uptake by phagocytes. Because of their persistence in circulation and the ability to be filter sterilized, ABC micelle formulations have been used in parenteral therapies. The ABC micelles are small enough to circulate freely within the bloodstream. Certain ABC micelle formulations with doxorubicin have been shown to accumulate in tumors in the body where they then release the passenger drug (M. Yokayama et al. (1999) J. Drug. Targeting 7, 171-186).

The core of an ABC defines the cargo space for the passenger compound, e.g., a hydrophobic therapeutic compound. There are three main categories of core forming blocks used for ABCs: Poloxamers, poly(L-amino acids) and poly (esters). Poly (ester) blocks are advantageous in their biocompatibility and biodegradability; examples include poly(ethylene glycol)-b-poly(lactic-co-glycolic acid), poly(ethylene glycol)-b-poly(D,L lactic acid), poly(ethylene glycol)-b-poly(glycolic acid, and poly(ethylene glycol)-b-poly(caprolactone). An exemplary PEG poloxamer used in pharmaceutical preparations is poly(ethylene glycol)-b-poly(propylene glycol)-b-poly(ethylene glycol). PEG copolymers based on the L-amino acids include, without limitation, poly(ethylene glycol)-b-poly(P-benzyl-L-glutamate), poly(ethylene glycol)-b-poly(L-lysine acid), poly(ethylene glycol)-b-poly(aspartic acid, poly(ethylene glycol)-b-poly(β-benzyl-L-aspartate), and acyl esters of the foregoing block copolymers. See also U.S. Pat. No. 5,449,513, which lists additional amphiphilic polymers as well.

An example of a hydrophobic therapeutic is fenofibrate, which modulates lipid metabolism. Its hydrophobic character has made suitable formulations for oral administration difficult; further examples include steroids such as prednisolone, antibiotics, antivirals, neurotropic agents, hormones, e.g. dihydrotestosterone, anti-inflammatory drugs such as indomethacin, anticonvulsants such as haloperidol dodecanoate, among others.

Anticancer agents include hydrophobic molecules such as paclitaxel, doxorubicin, geldanamycin, photosensitizers, daunomycin, among others. These compounds have a certain level of toxicity toward normal tissues and cells. There is a need for pharmaceutical formulations which are stable, minimize impact on normal tissue and function and which provide for advantageous release in target tissues.

Amphiphilic PEG-block-poly(L-amino acid) (PEG-b-PLAA) may form micelle structures that effectively encapsulate water-insoluble drugs (G. S. Kwon et al. (1994) Colloids & Surfaces B: Biointerfaces 2, 429-434; K. Kataoka et al. (2000) J. Control. Release 64, 143-153; M. Yokoyama et al. (1998) J. Control. Release 55, 219-229). PEG-b-PLAA micelles are may possess nanoscopic dimensions, shell of PEG, and nonpolar core of PLAA, which can take up and “protect” water-insoluble drugs. A primary advantage of PEG-b-PLAA is the potential for encapsulation of drugs by chemical or physical means inside the core of the micelles, consisting of PLAA blocks (M. Yokoyama et al. (1992) Bioconjugate Chem. 3, 295-301; Y. Li and G. S. Kwon (1999) Colloids & Surfaces B: Biointerfaces 16, 217-226; A. Lavasanifar et al. (2000) J. Biomed. Mater. Res. 52 831-835). In either situation, it is possible to tailor the structure of a core-forming PLAA block in order to enhance properties of PEG-b-PLAA micelles for drug delivery (Y. Li and G. S. Kwon (2000) Pharm. Res. 17(5), 607-611).

There is a longfelt need in the art for methods and compositions for encapsulating poorly (water) soluble compounds for use in pharmaceutical, food, cosmetic and industrial formulations. Desirably the encapsulated materials are nanoscopic in size, thermodynamically and kinetically stable, protect the hydrophobic compounds from self-aggregation and provide advantageous release rates. In the case of ABC formulations, there is desirably relatively low toxicity.

### SUMMARY OF THE INVENTION

The present invention provides methods for formulating hydrophobic “passenger” compounds, such as pigments, flavoring agents, insecticides, insect repellants, scent compounds, cosmetics, UV blocking agents, fungicides and therapeutic compounds including, but not limited to, anticancer agents such as paclitaxel, adriamycin or an alkyl derivative of cis-platin, a hydrophobic metabolic regulatory agent such as fenofibrate, and polyene antibiotics, especially amphotericin B, such that toxicity is reduced and so that the hydrophobic compound is readily and stably dispersed in aqueous formulations. The heart of the present invention is a cosolvent evaporation method for producing micelles comprising an amphiphilic block copolymer and a hydrophobic passenger compound, and optionally further comprising a lyoprotectant. The steps of producing the ABC micelles of the present invention include dissolving the passenger compound and the ABC in a volatile organic solvent, and then adding water to the miscible solution, with mixing, to promote the formation of micelles and the partitioning of the passenger compound into the micelle cores. The water content is greater than the cwc. Subsequently, the organic solvent is removed by evaporation under reduced pressure or elevated temperature. After loading, the ABC micelles can be freeze dried for later reconstitution.

The solvent useful in the method of the present invention is effective for dissolving the passenger compound and both the hydrophilic and the hydrophobic blocks of the ABC (taken separately), and the solvent desirably has a boiling temperature lower than that of water. It desirably forms an azeotrope with water. Examples include, without limitation, methanol, ethanol, propanol, isopropanol, acetonitrile and acetone. Where the solvent and water form an azeotrope, the azeoptropic mixture can be dried by removing the azeotrope under conditions of decreased pressure and/or elevated temperature.

The water is added to the ABC-passenger-solvent solution to a point greater than the cwc. The water content is sufficient that the hydrophobic portions of the copolymer interact with the result that the hydrophilic blocks of the ABC become directed to the outside and the hydrophobic blocks are directed to the interiors of the ABC micelles, together with the passenger compound, in the form of (loaded) ABC micelles.

Desirably, the passenger compound is a hydrophobic compound with a relatively flexible structure. Advantageously, the molecular weight is less than about 1500, preferably less than 1000. Exemplary flexible hydrophobic molecules include, but are not limited to, fenofibrate, and AmB; taxol is an exemplary molecule of intermediate flexibility, and griseofulvin is a rigid (not flexible) hydrophobic compound. Other passenger compounds for use in the methods of the present invention include, but are not limited to aliphatic derivatives of cis platin, acylated prodrugs, neurotropic agents, anticonvulsants, steroids, hormones, anti-inflammatory agents, anticancer therapeutics, geldanamycin and photosensitizers. For nonpharmaceutical use, the passenger compound can be a flavoring agent, a pigment, a fungicide, an insecticide, scented compound or other hydrophobic compound of interest.

Amphiphilic block copolymers useful in preparing micelles as described herein include those approved for veterinary and human pharmaceutical use. Specific examples include, but are not limited to PEG-b-poly (ester)s, PEG-b-poly(ε-caprolactone) or PEG-b-poly (L-amino acid) copolymers such as PEG-b-poly(N-hexyl-L-aspartamide) acyl esters. Others will be recognized for use in applications other than medical or veterinary use. Typically the polymer is dissolved at a concentration from about 0.01 to about 5 percent (w/v), desirably from about 0.1 to about 0.5 percent, and the passenger compound is typically added to the solvent-polymer solution in amounts ranging from about 0.1 to about 50% (weight passenger compound/weight polymer), desirably about 1 to about 30%, advantageously about 10%, prior to the addition of water. The cwc value typically ranges from about 1 to about 50%, often from about 5 to about 20% water, depending on the particular polymer and its initial concentration in the solvent. Water is added beyond the cwc, and the initial organic solvent is then removed by evaporation under reduced pressure or increased temperature. Subsequently, the aqueous solution of ABC micelles loaded with passenger compound is freeze dried. The ABCs can be used in the preparation of micelle preparations for use in aqueous systems, for pigment dispersal, for use in the fabric, food, art, cosmetic, agricultural, paper printing, paint and other industries. For the fabric, industrial and art applications, the polymers can include those which have the desirable properties of micelle formation and micelle complexation with the hydrophobic compound of interest.

The lyoprotectant acts to stabilize the micelles loaded with the passenger compound during the freeze drying process, so that the micelles do not coalesce with the result that the dried product does not readily disperse when an aqueous dispersant is added. The lyoprotectant can be a saccharide or polyol, for example, trehalose, sucrose or raffinose, or other hydrophilic polyol such as maltodextrin, fructose, glycerol, sorbitol, inositol and mannose, among others, with the proviso that the lyoprotectant must remain amorphous (and not crystalline) when dried. PEG works as well; lyoprotectants can be materials other than sugars. Typically, the ratio of polymer to lyoprotectant is from about 1:1 to about 1:50, preferably from about 1:1 to about 1:10, advantageously 1:5 on a weight:weight basis. The lyoprotectant can be added to the solvent along with the passenger compound and the polymer or it can be added after the addition of water. The aqueous micellar solution containing lyoprotectant can be dried under conditions of reduced pressure, for example, by freeze drying (lyophilization).

The dried material then contains micelles which are readily dispersed in aqueous milieus, water or solutions such as 5% dextrose or normal saline, in the case of human or veterinary applications. It is understood that the temperature used to remove the liquids is lower than the decomposition (or inactivation) temperature of the passenger compound.

In one specifically exemplified embodiment, the passenger compound is the polyene antibiotic amphotericin B (AmB) encapsulated within PEG-b-poly(N-hexyl-L-aspartamide)-acyl ester micelles. A general chemical structure for the final block copolymer products is given in FIG. 1, where n can be from 8 to 28, desirably 12 to 18 and preferably 16 (where the substituent is a stearoyl moiety). z can be from about 100 to about 300, and as specifically exemplified, z is 273. 25-x is from 1 to 5. As specifically exemplified there are x and 25-x subunits; however, the range can be from 15 to 40 total subunits, and x is from 5 to 90, advantageously 10 to 50. Desirably the level of acyl substitution is from about 70% to about 95%. The present core-forming polymer block improves the loading of the ABC micelles with polyene antibiotics, especially AnflB, and the use of a stearate acyl unit provides for stability of the micelle compositions, resulting in reduced toxicity and controlled release of antibiotic from the micelle in aqueous environments. The micelles loaded with AmB produced by the methods of the present invention are improved with respect to (reduced) toxicity and a reduction in the aggregation state of AmB and slow release in vivo.

Useful methods of drug loading for polymeric micelles result in high drug loading (typically 10% w/w), permit the use of hydrophobic ABCs for micelle formulations of poorly water-soluble drugs, permit sterilization, are easily scaled up for use in humans, and minimize or limit the use of unsafe organic solvents. Hydrophobic ABC with core forming blocks based on poly(ester)s that have large hydrophobic blocks (>1,000 gmol⁻¹) are attractive for drug delivery, owing to their high capacity for encapsulation of poorly water-insoluble drugs relative to hydrophilic ABCs (<1000 gmol⁻¹), which have smaller cores, and their stability that may permit sustained drug release and enhanced drug efficacy.

Based on these criteria, we have developed a novel method of loading polymeric micelles with hydrophobic passenger compounds. In one example, a poly(ester) based ABC and a poorly water-soluble passenger compound are dissolved in a volatile organic solvent, such as acetonitrile, that is miscible in water at all proportions. Water is then added slowly to induce micellization through the critical water content of the ABC (level of water required for assembly of ABC micelles). Water addition is continued until a level is reached that will allow for the evaporation of the organic solvent or azeotrope, resulting in ABC micelles that have encapsulated drug. The evaporation step leaves solely an aqueous solution. This solution can be filtered, freeze-dried, and stored in the solid state (after the addition of a lyoprotectant such as a sugar prior to drying), and later reconstituted in water or an aqueous solution. The solution can be filter sterilized for parenteral use with an aqueous vehicle, where pharmaceutical use is intended.

The cosolvent evaporation method of drug loading has been specifically exemplified with PEG-b-poly(ε-caprolactone), PEG-b-PCL, methoxy poly(ethylene glycol)-block-(ε-polycaprolactone) (CH₃O—(CH₂—CH₂—O—)N—(CO(CH₂)₅—O—)M—CO(CH₂)₅—OH, where N is from 50 to 150 and M is from 10 to 175, with two poorly water soluble drugs, fenofibrate and amphotericin B. PEG-b-PCL is hydrophobic. The cosolvent evaporation method of the present invention provides higher loading than the dialysis method for PEG-b-PCL micelle formation with fenofibrate and amphotericin B. Three molecular weights of PEG-b-PCL were used in the experiments described herein. PEG-b-PCL (5000:4000) has a molecular weight 9000; N is 113 and M is 34. PEG-b-PCL (5000:1000) has a molecular weight 6000; N is 113 and M is 8. PEG-b-PCL (5000:2500) has a molecular weight 7500; N is 113 and M is 21. For PEG-b-PCL (5000:18000), the molecular weight is 23000; N is 113 and M is 156. The ABC micelles prepared by this method allow release of the passenger compound at an advantageous rate for these drugs and others.

In other specifically exemplified embodiments, the amphiphilic block copolymer is poly(ethylene glycol)-block-poly(ε-caprolactone) or other copolymer of poly(ester)s. The molecular weight of the poly(ethylene glycol) portion of the copolymer is from about 2000 to about 20,000, desirably from about 3500 to about 12000. The molecular weight of the hydrophobic core forming portion can be from about 800 to about 18,000, desirably from about 1000 to about 6000, desirably 4000 to 5000. The weight of the ABC is chosen, at least in part, according to the size and flexibility of the passenger compound.

ABC micelles are prepared by the present methods of the invention are loaded with hydrophobic therapeutic agents including polyene antibiotics including but not limited to AmB, anticancer agents such as paclitaxel and doxorubicin and fenofibrate, neurotropic agents and others, with advantageous properties of toxicity, solubility in aqueous milieus and release rate in vivo.

Using the cosolvent evaporation method of the present invention, we prepared a drug loaded solid that easily reconstitutes to produce drug loaded ABC micelles in solution, when a lyoprotectant is present before drying. After the encapsulation of fenofibrate, the aqueous PEG-b-PCL micelles can be freeze-dried in the presence of a lyoprotectant, such as a sugar, stored in the solid-state to prevent drug loss and degradation of the drug, and easily reconstituted with water, buffer or other aqueous medium prior to use. Because fenofibrate has been well characterized in the solid-state with PEG solid dispersions, solid analysis was performed to further examine the present micelle formulation. Powder X-ray diffraction was performed at 25° C. on a Scintag, Inc DMC-008 (Cupertino, Calif.) and analyzed with Scintag software.

Paclitaxel-loaded PEG-b-PCL micelles were prepared in the same way as described for fenofibrate-loaded micelles. Similar properties are observed for the paclitaxel-ABC micelles as for the fenofibrate-loaded ABC micelles; thus, the methods of the present invention are applicable to various hydrophobic passenger compounds, especially those of pharmaceutical importance as well as compounds useful for other purposes.

It was necessary to prevent polymer crystallization during drying, or in the solid-state. Sucrose, raffinose pentahydrate, and trehalose dihydrate served as lyoprotectants; others may be apparent to the skilled artisan. Sugars were added to the micellar mixture prior to solvent or azeotrope evaporation. The ACN-water mixture was at 70% water and the sugars were soluble at this proportion. The compositions of sugars tried were sugar to polymer ratios of 0, 5, 10, 25, 50, and 125 (w/w). After the solvent was evaporated the clear solution was freeze dried on a Labconco freeze-drying system until dry (approximately 24-36 hours).

The solid samples were then subjected to X-ray diffraction. As the amount of sugar added to the formulation increased, the X-ray profiles reflected decreases in the magnitudes of the PEG and PCL crystalline peaks. For example, in examining PEG-b-PCL (5000:4000) with the presence of sucrose in 5, 10, 25, 50 times amount of polymer, the PEG and PCL peaks were diminished. When a sucrose to polymer w/w ratio of 25:1 was used, the polymer crystal peaks were no longer apparent in the powder X-ray diffraction analysis. Samples prepared with the presence of raffinose pentahydrate and trehalose dihydrate no longer showed crystallinity with a sugar to polymer ratio of 25:1. Without wishing to be bound by theory, this is believed to be due to the sugar inhibiting crystallization of the PEG upon freeze-drying.

Another technique to examine ABC micelle shape and size as a solid is transmission electron microscopy (TEM). Briefly, the polymeric micelle sample with and with out the presence of drug was mixed with negative stain, ammonium molybdate, with the ratio of micelle solution to stain of 10:1. A drop of the mixed stain and sample was added to a coated grid. The solution was allowed to air dry, and excess liquid was removed using filter paper. The grid was then imaged using a Philips CM120 electron microscope.

TEM showed evidence of nanoscopic spherical micellar structures. PEG-b-PCL (5000: 1000) had the most uniform small sized structures of the three compositions. The PEG-b-PCL (5000:4000) micelles dried without lyoprotectant appeared aggregated and polydisperse. However for micelles prepared with lyoprotectant, PEG-b-PCL (5⁰00:4000)'s micellar polydispersity and size were diminished, as determined by TEM. The amount of trehalose used was roughly 50 times the amount of PEG-b-PCL (5000:4000), i.e., a sugar to polymer mol/mol ratio of about 800:1. At this amount of trehalose, the micelles were readily reconstituted from solution. Other lyoprotectants, including but not limited to, sucrose and raffinose offer similar stabilization of micellar structure during the drying process. It is a matter of routine to optimize the proportions of lyoprotectant to polymer when the polymer or the lyoprotectant is varied from the specific example provided herein.

The present invention further encompasses ABC micelles formed by the cosolvent evaporation method which encapsulate AmB, other polyene or other therapeutic compound such as paclitaxel, or other poorly water soluble passenger compound. Also within the scope of the present invention are freeze-dried preparations of the ABC micelles of the present invention, as set forth above, especially those comprising a polyene such as AmB or comprising fenofibrate. Also within the scope of the present invention are reconstituted ABC micelles of the present invention, especially AmB-loaded or fenofibrate-loaded ABC micelles, reconstituted in 5% sterile dextrose solution.

### BRIEF DESCRIPTION OF THE DRAWINGS

FIG. 1 provides the general chemical structure of PEG-b-p(6-HHA)-acyl ester conjugates used in the present methods. The final block copolymer products are named as PEG-b-poly(N-hexyl-L-aspartamide-Z-acid) where Z designates the acyl moiety of the esterified chain, i.e. acetic (A), hexanoic (H), lauric (L), or stearic (S), and x corresponds to the average number of acyl groups attached per 25 L-Asp as determined by ¹H NMR. For simplicity, the polymers are abbreviated as PEG-b-p(HAZA), PEG-b-p(HAAA), PEG-b-p(HAHA), and so on.

FIG. 2 illustrates solvent/environmental effects on the typical four-peak AmB absorption spectrum.

FIG. 3 is the absorption spectrum of amphotericin B encapsulated in PEG-b-p(6-HHA) and PEG-b-p(HAZA) as a function of acyl composition. Polymer concentrations are between 0.0625 and 0.125 mg/mL.

FIG. 4 provides the time-dependent hemolytic profile of AmB (ca. 3 μg/mL) encapsulated in PEG-b-p(6-HHA) and PEG-b-p(HAZA) micelle formulations.

FIG. 5A shows the absorption spectrum of AmB encapsulated with PEG-b-p(HAA) or PEG-b-poly(HASA) as a function of the degree of stearate esterification. FIG. 5B provides the absorption of AmB encapsulated with PEG-b-p(HASA) micelles as a function of drug levels.

FIGS. 6A-6B show the dose dependence of hemolysis for free AmB (FIG. 6A) and AmB encapsulated by PEG-b-p(HASA) micelles (FIG. 6B). Hemolysis of bovine erythrocytes (7.4×10⁷ cells/mL in PBS) was measured over time.

FIGS. 7A-7B demonstrate the effectiveness of PEG-b-HASA (91%) micelle formulations of AmB in a neutropenic mouse model of disseminated candidiasis. FIG. 7A compares three doses (as AmB) of the AmB loaded micelles; FIG. 7B compares the same doses of AmB (Fungizone™, E. R. Squibb & Sons, LLC, Princeton, N.J., sodium deoxycholate-solubilized AmB).

FIG. 8A shows the solubility of fenofibrate in the presence of preformed PEG-b-PCL micelles and PEG 8000. FIG. 8B shows the solubility of fenofibrate in water-acetonitrile mixtures.

FIG. 9 illustrates the maximum amount of fenofibrate encapsulated into PEG-b-PCL micelles with the cosolvent evaporation method described herein.

FIG. 10 illustrates the fenofibrate loading efficiency in PEG-b-PCL micelles using the cosolvent evaporation method.

FIGS. 11A, 11B and 11C shows the radius of hydration, as measured by dynamic light scattering (DSL) analysis of PEG-b-PCL particles as a function of water content, for PEG-b-PCL 5000:4000, 5000:25000, 5000:1000, respectively. The initial hydrodynamic radius of hydration of PEG-b-PCL (5000:4000) at 0.1% w/w was 2.65 nm.

FIGS. 12A-12B show the average micelle particle size of the fenofibrate-loaded PEG-b-PCL micelles before (FIG. 12A) and after freeze drying (FIG. 12B).

FIG. 13 summarizes the cosolvent evaporation process for preparation and subsequent use of loaded micelles prepared with amphiphilic diblock polymers as exemplified for a drug. The ABC and passenger compound are first dissolved in a volatile organic solvent which is miscible with water and may form an azeotrope with water. Water is added to drive micellization, and after sufficient partitioning of passenger compound into ABC micelles, the azeotrope (or solvent) is removed through evaporation, desirably at reduced pressure to provide an aqueous solution of the loaded micelles. The ABC micelles can be dried (freeze-dried) for storage and later reconstituted with water or an aqueous solution.

### DETAILED DESCRIPTION OF THE INVENTION

As used in the context of the present invention, a solvent is a liquid used to produce a solution with at least one amphiphilic polymeric material and at least one “passenger compound”. For use in the present methods, the solvent is one which desirably has a boiling temperature lower than that of water (less than 100° C at 1 atm). The solvent forms an azeotrope with water, advantageously a negative azeotrope.

As used herein, a passenger compound is a hydrophobic compound (not freely soluble in water) which is encapsulated within an amphiphilic polymeric material using the methods of the present invention. It can be a pigment, a flavoring agent, or a therapeutic compound useful in human and/or veterinary medicine. Typically, the molecular weight of the passenger compound is less than about 1000 d. Desirably, the structure of the passenger compound is flexible so that hydrophobic interactions between the passenger compound and the hydrophobic portion (also flexible) of the polymeric material allow good loading in the ABC micelles. As taught herein, the micelles desirably have a diameter of from about 10 nm to about 200 nm, desirably from about 15 to about 75 nm.

Other hydrophobic therapeutic molecules used as passenger compounds in the methods of the present invention include, without limitation, paclitaxel, acylated cis platin, cancer chemotherapeutic agents, and others. For nonpharmaceutical use, poorly water soluble passenger compounds can include, but are not limited to, flavoring agents, scent compounds, pigments, dyes, fungicides, insecticides, insect repellants, ultraviolet light absorbing compounds and others

Fenofibrate, 2-[4-(4-chlorobenzoyl)phenoxy]-2-methylpropanoic acid 1-methylethyl ester, molecular weight 360.83, is a compound used in humans as a therapeutic for regulating lipid metabolism. It is insoluble in water, and it is characterized by a relatively flexible molecular structure. It has been successfully encapsulated with PEG-PCL using the methods of the present invention (see, e.g., below).

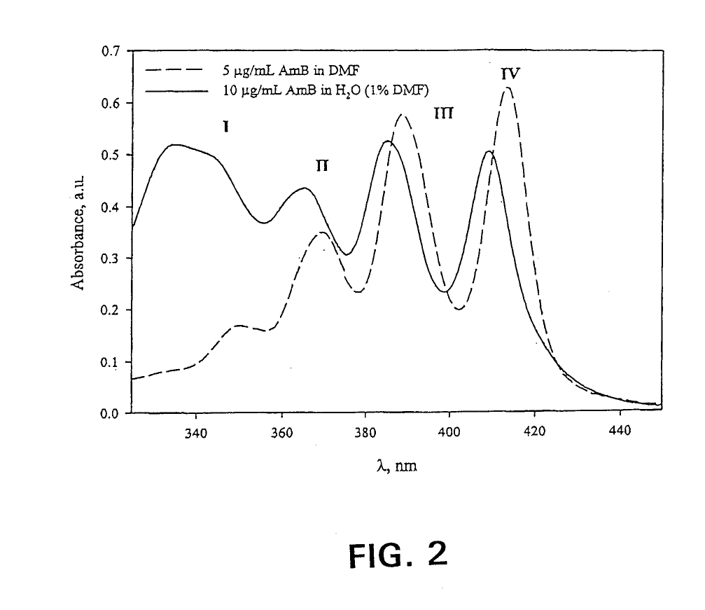

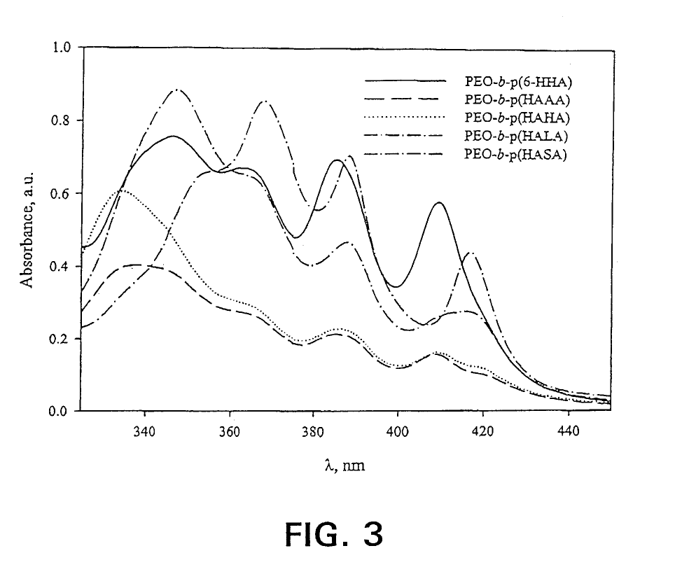

With seven conjugated double bonds, AmB is a very strong chromophore and is easily detected by UV-Vis spectroscopy. Its absorption spectrum usually contains four primary peaks (FIG. 2). Although the positions and intensities of the absorption bands vary with respect to the local environment, the ratio of the first (346 nm) to the fourth (409 nm) peaks, I/IV, can be taken as a measure of relative aggregation state. This ratio can be quite low (<0.25) for nonaggregated, or monomeric, AmB and as high as 2.0 for highly aggregated drug (Barwicz, J. et al. (1992) Antimicrob. Ag. Chemother. 36, 2310). Under the experimental conditions, the levels of encapsulated drug and the encapsulation efficiency were similar for all of the polymer/AmB formulations (Table 2). The absorption spectrum of AmB encapsulated by PEG-b-p(N-HA)-acyl conjugates is given in FIG. 3. The effects of acyl chain length are quite apparent. The absorption spectrum of AmB encapsulated by PEG-b-p(6-HHA) contains four absorption bands centered at 346, 362, 385, and 409 nm with a I/IV ratio of 1.2. Although this value indicates a low level of AmB aggregation compared to some of the micelle formulations, the concentration of PEG-b-p(6-HHA) in the formulation is much greater than for the acyl conjugates. Previously, we found that the CMC of PEG-b-p(6-HHA) micelles is substantially high (approximately 0.5 mg/mL) compared to the acyl conjugates which had CMCs on the order of 20 μg/mL (Adams, M. L. et al. (2002) J. Biomat. Sci., Polym. Ed. 13, 991). Therefore, PEG-b-p(6-HHA) was reconstituted to 10 mg/mL in order to ensure that the polymer concentration was well above the CMC. Consequently, the drug to polymer ratio for this formulation is quite low (Table 2) compared to those of the PEG-b-p(HAZA) formulations. Because the CMC is related the thermodynamic equilibrium that exists between the micelles and unimers, or thermodynamic stability, we hypothesized that PEG-b-p(6-HHA) might not be stable enough to withstand dilution. Therefore, even with low levels of drug aggregation, this formulation might not be a suitable delivery system for AmB. In fact, the formulation did not maintain integrity during GPC analysis. All of the acyl conjugates, with and without AmB, formed micelles on the order of 106 g/mol (data not shown), which was consistent with previous results for stearate ester micelles (Lavasanifar, A. et al. (2001) J. Contr. Rel. 77, 155). However, PEG-b-p(6-HHA) micelles did not remain intact on the column and could not be detected. As expected, PEG-b-p(6-HHA) did not withstand dilution.

The absorption spectrum of AmB encapsulated in PEG-b-p(N-HA)-acyl conjugate micelles varies considerably (FIG. 3). In PEG-b-p(HAAA), PEG-b-p(HAHA), and PEG-b-p(HALA) micelles, encapsulated AmB appears to be quite aggregated. In contrast, the spectrum observed for the PEG-b-p(HASA) formulation is distinct from those of the other acyl conjugate formulations. It is worth noting that the AmB/PEG-b-p(HASA) spectrum shares three bands with the AmB/PEG-b-p(HALA) spectrum in which the absorption bands are red-shifted compared to the shorter acyl chain formulations. The λₘₐₓ of the AmB/PEG-b-p(HALA) and AmB/PEG-b-p(HASA) formulations are is 347 and 368 nm, respectively. For the shorter chain lengths, λₘₐₓ is on the order of 331-338 nm. The peak at the highest wavelength in the absorption spectrum of the AmB/polymer formulations is also of particular interest. For the AmB/PEG-b-p(HALA) and AmB/PEG-b-p(HASA) samples, the last absorption band lies at 416 and 417 nm, respectively, compared to 409 nm for the shorter acyl chain length formulations. The spectrum of the AmB/PEG-b-p(HASA) formulation is quite similar to that observed with AmB is complexed with ergosterol, while the AmB/PEG-b-p(HALA) spectrum is similar to that observed when AmB is complexed with cholesterol (Charbonneau, C. et al. (2001) Biophys. Chem. 91, 125; Gruda, I. et al. (1988) Biochem. Cell Biol 66, 177). Due to striking similarities between our spectra and those in the literature, we propose that the interactions between AmB and PEG-b-p(HALA) are weaker than those between AmB and PEG-b-p(HASA). Without wishing to be bound by theory, it is believed that increasing acyl chain length results in stronger drug-polymer interactions, whereas AmB self-association predominates at lower acyl chain lengths. Furthermore, increased interaction between the polymer side chains and AmB results in a more gradual or sustained release profile.

Polymeric micelles were prepared and stored in the solid state. Upon reconstitution with 1.0 mL of phosphate buffered saline, PBS, all formulations yielded clear solutions. The reconstituted solutions contained 0.5 and 10.0 mg/mL of PEG-b-p(HASA) and PEG-b-p(6-HHA), respectively. The critical micelle concentration (CMC) is the concentration at which micelle formation begins in aqueous solution and is related to the thermodynamic equilibrium between micelles and unimers, i.e. thermodynamic stability. A twenty-fold excess of PEG-b-p(6-HHA) compared to PEG-b-p(HASA) was used for AmB encapsulation in order to account for the differences observed previously in the magnitudes of the CMCs and ensure micelle formation (Adams et al., 2002, supra). In addition, structure-property studies conducted with poly(ethylene glycol)-block-poly(N-hexyl-L-aspartamide)-acyl conjugate micelles indicated that PEG-b-p(6-HHA) micelles contain more fluid core regions than those assembled from the esterified diblock copolymers (Adams et al., 2002, supra) Therefore, PEG-b-p(6-HHA) micelles might be less kinetically stable than micelles formed by the esterified polymers. Due to decreased thermodynamic and kinetic stability, it is reasonable to expect that micelles prepared from PEG-b-p(6-HHA) might dissociate rapidly upon dilution. While it was possible to approximate the molecular weight for the PEG-b-p(HASA) micelles by GPC, PEG-b-p(6-HHA) micelles were indeed unstable and did not remain intact on the column (Table 4). Esterification of PEG-b-p(6-HHA) with stearic acid at 46 and 91% substitution imparted the stability necessary to withstand dilution on the column (Table 4). Generally, the molecular weight of polymeric micelles as determined by GPC analysis is on the order of 106 g/mol (Yamamoto, Y.; Yasugi, K.; Harada, A.; Nagasaki, Y.; Kataoka, K. (2002) J. Contr. Rel. 82, 359).

For all esterification levels studied (0, 46, and 91%), AmB was encapsulated with similar loading efficiency. For the acylated polymers, the drug level is substantially higher than that achieved in previous efforts, which resulted in approximately 0.2-0.9 AmB:polymer on a mole to mole basis (Lavasanifar et al. (2002) Pharm. Res. 19(4), 418). Improved assembly conditions are likely responsible for the increased drug to polymer ratios. Although the final AmB concentration in each formulation in the present study was similar, the AmB:polymer molar ratio is very low (approximately 0.1) for PEG-b-p(6-HHA). These results indicate that PEG-b-p(6-HHA) micelles have a low capacity for AmB solubilization.

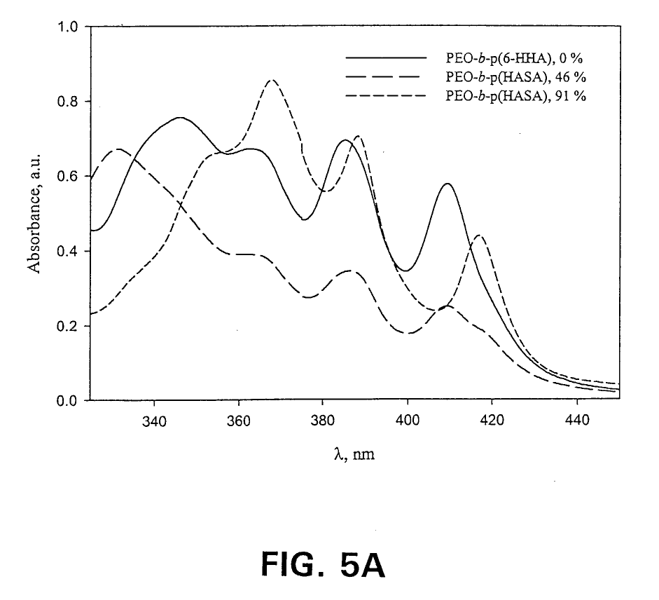

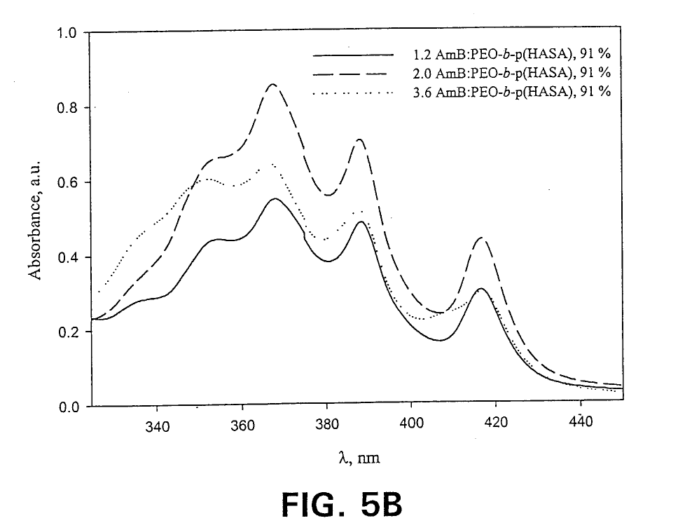

Because all of the copolymers used in the present study have the same 12,000 g/mol PEG hydrophilic block, 25 repeat-unit poly(L-Asp) backbone, and hexyl spacer, the differences between the formulations may be attributed to relative level of stearate attachment. The absorption spectrum of AmB encapsulated by PEG-b-p(6-HHA) and PEG-b-p(HASA), 46 and 91% is given in FIG. 5A. The absorption spectrum of AmB encapsulated in the unesterified PEG-b-p(6-HHA) micelles is characterized by four bands at 346, 362, 385, and 409 nm. In addition, the I/IV ratio is 1.2 and indicative of a reduction in the self-aggregation state of the drug.

The effects of stearate conjugation on the relative aggregation state of AmB are quite apparent. For polymeric micelles containing 46% stearate attachment, the absorption spectrum of encapsulated AmB is characterized by strong absorption in the 330-340 nm region and indicative of highly self-aggregated species (FIG. 5A). However, the fourth absorption band in the spectrum contains a shoulder at higher wavelengths. Hence, there may be a small quantity of less self-aggregated drug species. At 91% esterification, the absorption spectrum of encapsulated AmB is characterized by peaks at 354, 368, 388, and 417 nm (FIG. 5A). This finding confirms previous studies, which indicated that increased esterification with stearate side chains results in a reduction in AmB self-aggregation (A. Lavasanifar et al. (2002) Pharm. Res. 19, 418). Furthermore, the λₘₐₓ is red-shifted to 368 nm, and the absorption bands are narrower than those observed when AmB is encapsulated by PEG-b-p(6-HHA) and PEG-b-p(HASA), 46%. This spectrum bears striking similarity to that observed when AmB is complexed with ergosterol (D. Andes et al. (1999) Antimicrob. Ag. Chemother. 43, 2116; I. Gruda et al. (1988) Biochem. Cell Biol. 66, 177). Therefore, we postulate that AmB may interact strongly with the stearate side chains of micelles prepared from highly substituted PEG-b-p(HASA).

The effects of initial loading conditions on the final AmB level in PEG-b-p(HASA), 91% micelles are given in Table 5. The corresponding absorption spectra are given in FIG. 5B. For lower AmB:PEG-b-p(HASA), 91% ratios (1.2 and 2.0), the absorption spectrum is very similar to that obtained from AmB/ergosterol complexes as mentioned above. However, at a loading level of 3.6 AmB:polymer, an increase in absorption intensity is observed in the 330 nm region of the spectrum. In addition, the absorption bands are broader than those of the other PEG-b-p(HASA), 91% formulations. Taken together, these spectral features indicate a possible threshold level for the interaction of AmB with the stearate side chains of PEG-b-p(HASA) micelles. It is possible that AmB begins to self-associate at high levels of drug-loading.

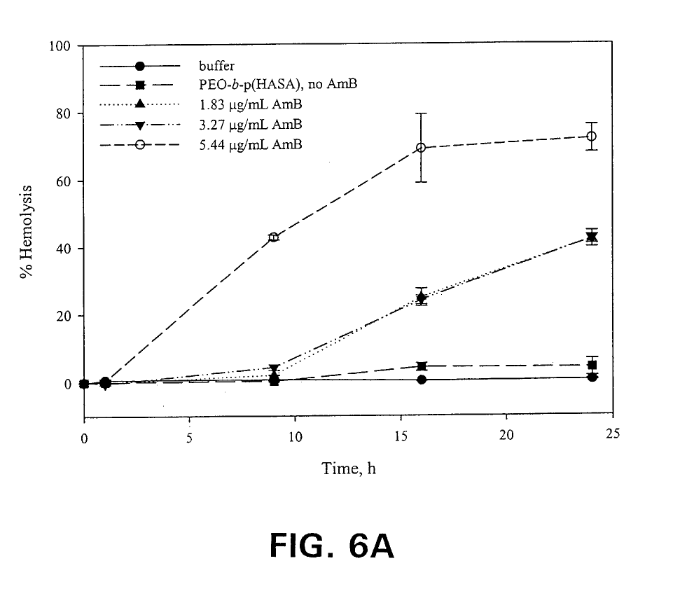

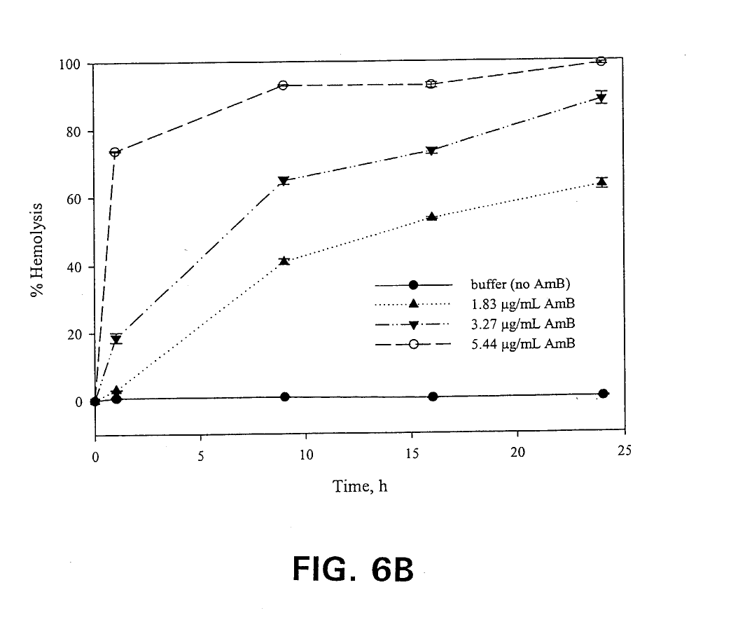

Hemolytic profiles of the polymeric micelle formulations indicate time-dependent activity and reflect sustained drug release. For the formulations containing PEG-b-p(6-HHA) or PEG-b-p(HASA), 46% micelles in the absence of AmB, no hemolysis was observed over the course of 24 h. However, slight hemolysis (approximately 4%) was observed in the presence PEG-b-p(HASA), 91% after 16 h. Therefore, the leakage of hemoglobin from the bovine erythrocytes is attributed to action of AmB on the cell membranes. At similar drug levels (approximately 3 μg/mL), the onset of hemolysis is more gradual for the PEG-b-p(HASA), 91% formulation than for those with 0 and 46% stearate attachment (FIG. 6). When taken in conjunction with the corresponding absorption spectra, this finding supports interaction between the stearate side chains of PEG-b-p(HASA) and AmB.

Free AmB caused 40% hemolysis after 1 h and nearly 80% hemolysis after 9 h (FIG. 6). The onset of hemolytic activity was delayed upon encapsulation in all of the polymeric micelles studied, regardless of the aggregation state of the drug. Although PEG-b-p(6-HHA) encapsulation seems to lead to relatively low levels of drug self-aggregation as evidenced by the corresponding absorption spectrum (FIG. 5A), hemolytic activity is readily apparent. Such rapid hemolysis is likely due to instability of the PEG-b-p(6-HHA) micelles. As discussed previously, GPC studies indicated rapid dissociation of the PEG-b-p(6-HHA) micelle structures. Consequently, AmB-induced hemolysis occurs quickly.

Hemolytic profiles of the AmB/PEG-b-p(HASA), 46% formulation is similar to that observed with the unesterified polymer formulation. While 46% stearate esterification increased micelle stability as evidenced by GPC analysis, it had little impact on the time-dependent hemolytic activity of AmB. Absorption spectroscopy indicated that AmB was encapsulated by PEG-b-p(HASA), 46% in a self-aggregated state (FIG. 5A). Therefore, it is likely that the drug was released as an aggregated species, which would be capable of inducing rapid hemolysis of mammalian cells. On the other hand, AmB encapsulated at the same level by 91% esterified polymer had a markedly different hemolytic profile with 4% hemolysis at 9 h, 20% at 16 h, and 40% at 24 h (FIGS. 6A-6B). In this case, the absorption spectrum of encapsulated AmB (FIG. 5A) indicated that the drug was encapsulated in a nonself-aggregated state. Hence, it is reasonable to propose that the gradual build-up of hemolytic activity observed when AmB was encapsulated in highly substituted polymeric micelles is due to the sustained release of relatively nonaggregated species.

The onset of hemolysis for AmB encapsulated by PEG-b-p(HASA), 91% was delayed compared to equivalent concentrations of free AmB (FIG. 6B). At all concentrations studied, the onset of hemolysis is delayed when AmB is encapsulated in PEG-b-p(HASA), 91%. On the other hand, the level of drug-loading in PEG-b-p(HASA), 91% micelles did impact the time-dependent hemolytic profile. At lower drug levels (1.2 and 2.0 drug:polymer), the profiles are nearly identical with very little hemolysis at 9 h, 20% at 16 h, and approximately 40% at 24 h (FIG. 6B).

However, the onset of hemolysis is more rapid at an AmB:polymer loading level of 3.6. These findings correlate with the corresponding absorption spectra (FIG. 5B), which suggest that AmB self-aggregation begins to occur at high drug levels, i.e. 3.6 AmB:PEG-b-p(HASA), 91%. It is plausible that AmB is released slowly from PEG-b-p(HASA), 91% micelles. However, at high drug levels, AmB may be released as a more aggregated species or in quantities that more quickly surpass the threshold required for self-aggregation in aqueous media. As a result, toxicity toward mammalian cells, as evidenced by hemolysis of bovine erythrocytes, becomes apparent.

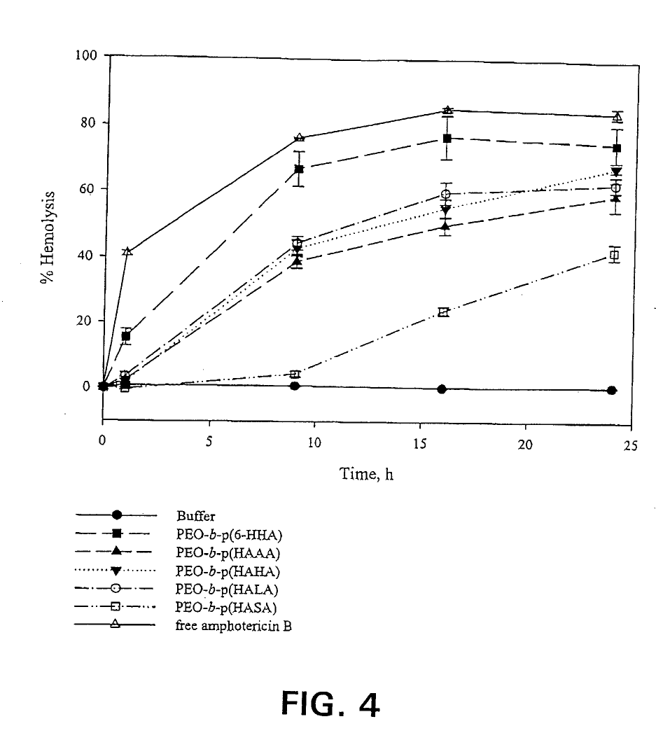

Hemolysis was not observed in “blank” polymer formulations containing no AmB (data not shown). However, very slight hemolysis (ca. 4%) was observed in the presence PEG-b-p(HASA) after 16 h. Consequently, the hemolytic activity observed in the presence of AmB/polymer formulations can be attributed to AmB. Incubation with free AmB results in immediate hemolysis, while encapsulated AmB exerts hemolysis more gradually (FIG. 4). It is possible that the relatively low toxicity of the acyl conjugate formulations observed toward bovine erythrocytes is the result of slow diffusion of AmB from the micelles. Free AmB caused 40% hemolysis after 1 h and 80% hemolysis at 16 h. A similar profile was observed for the less stable PEG-b-p(6-HHA) formulation which caused 15% hemolysis at 1 h. Furthermore, hemolysis builds over time in a manner similar to free AmB with 70% hemolysis at 16 h. Without wishing to be bound by theory, it is believed that AmB is released rapidly from PEG-b-p(6-HHA) micelles due to the relatively low stability indicated by the high CMC value. This explanation is in line with our GPC findings.

With increasing acyl chain length, in vitro hemolysis proceeds at a much more gradual rate (FIG. 4). Perhaps the steady increase in hemolysis reflects the sustained release of AmB from PEG-b-p(N-HA)-acyl conjugate micelles. The hemolytic profiles of the PEG-b-p(HAAA), PEG-b-p(HAHA), and PEG-b-p(HALA) micelle formulations are quite similar, with relatively little hemolysis after 1 h, approximately 40% at 9 h, and around 60% at 24 h. PEG-b-p(HASA) micelles have the most dramatic effect on AmB-induced hemolysis with only slight hemolysis (ca. 4%) at 9 h, approximately 20% at 16 h, and about 40% after 24 h. At the 24 h mark, the hemolytic activity of the PEG-b-p(HASA)/AmB formulation is approximately half that of free drug. We believe that interaction between encapsulated AmB and the stearate side chains indicated by the corresponding absorption spectrum results in slow, or sustained, drug release. In contrast, AmB encapsulated by PEG-b-p(HAAA), PEG-b-p(HAHA), and PEG-b-p(HALA) micelles interact to a lesser extent with AmB, enabling drug-induced hemolysis to proceed more readily.

Several factors may be responsible for observed time-dependent hemolytic activity of the AmB/polymer formulations. Perhaps the most obvious contribution is from micelle instability. Less stable micelles are believed to more rapidly dissociate, resulting in a relatively high concentration of AmB in solution. Indeed, this behavior is likely responsible for the similarities observed in the time-dependent hemolytic profile of AmB encapsulated by PEG-b-p(6-HHA) and that of free drug. Another important factor could be time-dependent release. In other words, the presence of a “lag phase” can be explained in the context of an AmB “threshold level” necessary for pore formation in the target cells (Forster, D. et al. (1988) J. Pharm. Pharmacol. 40, 325). A carrier vehicle may delay the onset of pore formation by limiting the diffusion of free AmB into the aqueous environment. If released AmB interacts to a greater extent with ergosterol-containing fungal cell membranes, a threshold concentration necessary for mammalian cell toxicity may not be reached. As AmB binds preferentially to ergosterol, slower release of AmB may help favor fungal cell selectivity. Without wishing to be bound by any particular theory, the PEG-b-p(N-HA) conjugate micelle family is believed to reduce the nonspecific toxicity of encapsulated AmB through such a mechanism. Furthermore, a carrier exhibiting an increased affinity for the drug reduces the equilibrium concentration of AmB in the aqueous phase. In this way, encapsulation by PEG-b-p(HASA) lowers the nonselective toxicity of AmB to a greater extent than the other polymers in the series.

It is unlikely that the effects of diffusion of AmB from the PEG-b-p(HAZA) micelles can be entirely separated from the relative aggregation state of encapsulated AmB. Specifically, whether AmB is released in a relatively monomeric or highly aggregated form has a dramatic effect on toxicity to mammalian cells. If AmB is encapsulated in a highly aggregated state and released in a similar state, the formulation should be toxic to both cell types with minimal specificity. In the present study, formulations containing highly aggregated AmB did indeed exhibit increased hemolysis over the apparently less-aggregated stearate formulation. Therefore, it is likely that the requirement of a threshold level will apply more closely to polymeric micelle drug-delivery systems in which AmB is encapsulated in a relatively nonaggregated state. For example, the onset of hemolysis in the PEG-b-p(HASA) formulation is delayed more than with the other acyl conjugate formulations. Eventually, however, hemolytic activity is apparent, reflecting the necessity of a threshold level of AmB in solution for effective pore formation. Then again, aggregation state is not the only consideration. Even though AmB was encapsulated in a fairly nonaggregated state by PEG-b-p(6-HHA) micelles, hemolysis occurred rapidly and in a similar manner to free drug. This observation indicates the need for relatively stable micelle structures in order for the aggregation state of encapsulated drug to serve as a predictor of hemolytic activity. Micelle stability, or low CMCs, will be very important for any formulation subjected to dilution in the in vivo environment. In summary, the toxicity of AmB to mammalian cells is reduced by sustained release of relatively nonaggregated AmB from stable micelle-like structures.

The interactions of AmB with itself, membrane sterols, and carriers are quite complex, yet the aggregation state of the drug is a good indicator of toxicity and hemolytic activity. Consequently, the ability to modulate the equilibrium between the different aggregates is of primary concern for AmB formulation development. The length of the esterified acyl chain in PEG-b-p(N-HA)-acyl conjugates has a profound influence on the aggregation state of encapsulated AmB. In turn, the relative aggregation state affects the hemolytic activity of AmB toward bovine erythrocytes. In particular, PEG-b-p(HASA) micelles have a pronounced effect on the time-dependent hemolytic profile of AmB. Through manipulation of the core properties, it is possible to design drug-delivery systems with properties tailored to the application at hand. Specifically, we can fine-tune the release of AmB from PEG-b-p(HAZA) micelles by adjusting the structure of the core-forming poly(L-Asp) block. Stearate esterification imparts the stability necessary for a long-circulating vehicle and leads to a decrease in toxicity in vivo through alteration of the equilibrium distribution of AmB in the aqueous phase.

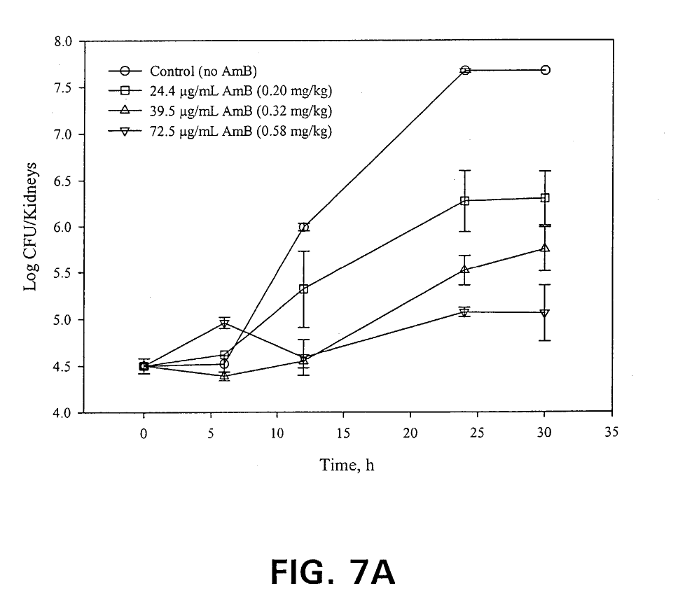

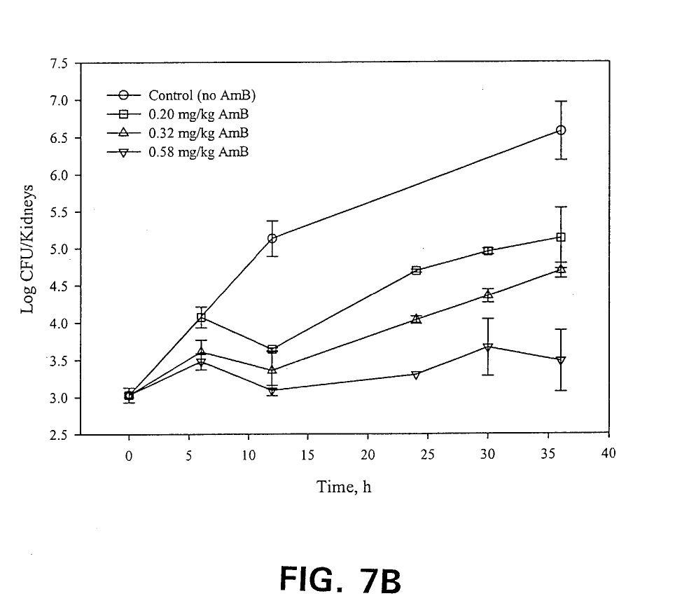

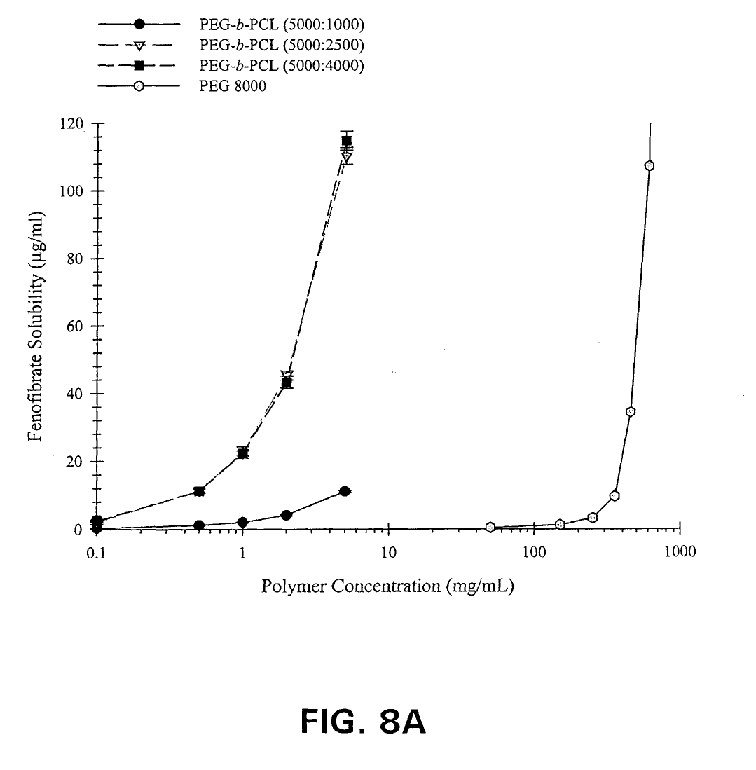

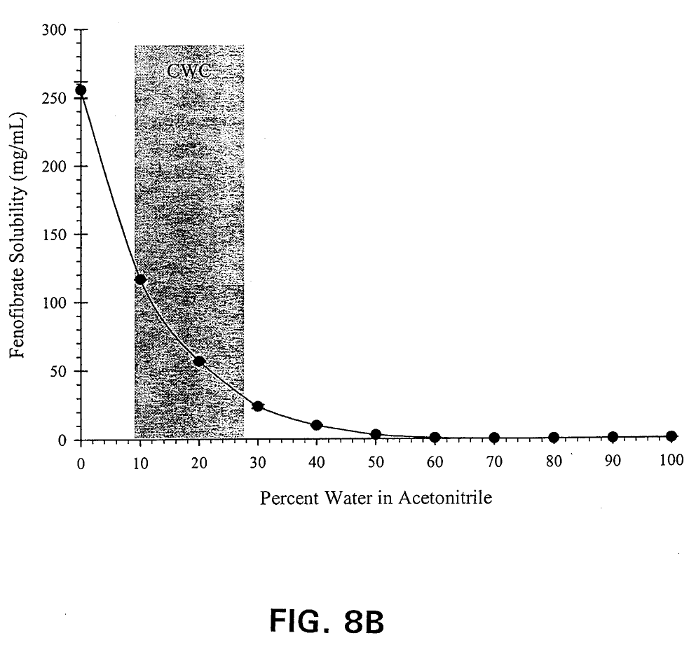

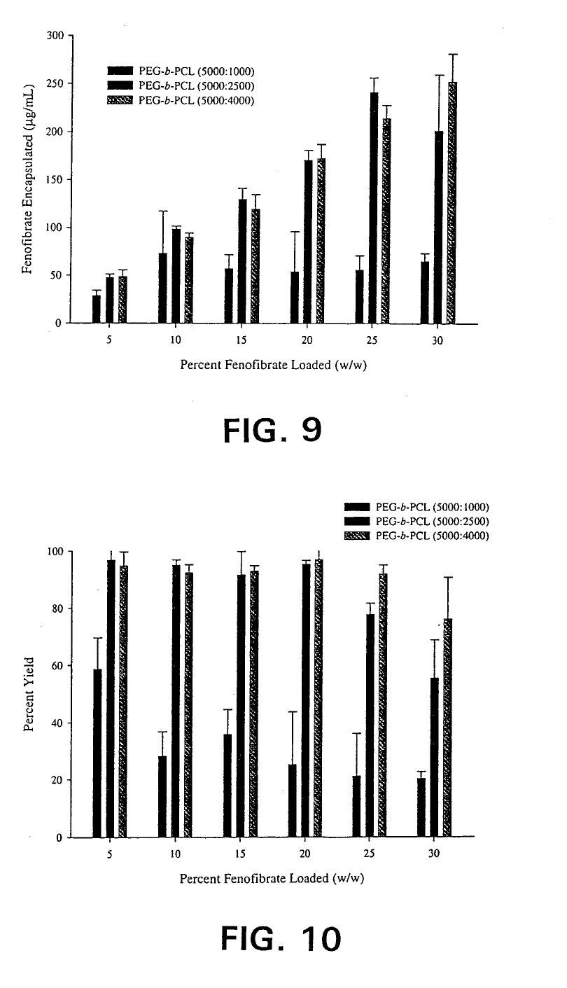

Fungal growth curves for both treated and control animals following single iv doses of AmB are given in FIGS. 7A-7B. Following tail vein inoculation, the growth of C. albicans in the kidneys of both treatment and control mouse groups increased (3.2-3.5 log CFU/kidney) over the time period of the studies. The infection level (CFU_{kidney}) is similar to that obtained in previous studies using the same murine model, which resulted in approximately 3.5-4.5 CFU_{kidney} two hours following inoculation (Andes, D. et al. (2001) Antimicrob. Agents Chemother. 45(3), 922; Andes, D. et al. (2000) Antimicrob. Ag. Chemother. 44(4), 938; Andes, D. et al. (1999) Antimicrob. Ag. Chemother. 43(9), 2116). Treatment with both AmB/PEG-b-p(HASA), 91% and AmB as Fungizone (sodium deoxycholate-stabilized AmB) suppressed organism growth in a dose-dependent manner at the doses studied. Kidney-CFU determination indicated that the efficacy of AmB encapsulated by PEG-b-p(HASA), 91% was not significantly different than that of Fungizone at doses of 0.2, 0.3, and 0.6 mg/kg of AmB (p=0.70). Hence, encapsulation of AmB by PEG-b-p(HASA), 91% micelles did not have a detrimental effect on the potent antifungal activity of AmB in a neutropenic murine model of disseminated fungal infection. These results contrast to those obtained for conventional liposomal AmB (AmBisome), of which higher doses are required to obtain equivalent efficacy to Fungizone (White, M. H. In Progress in the use and delivery of polyenes in antifungal therapy; Dixon, G. K.; Copping, L. G.; Hollomon, D. W., Eds.; Antifungal Agents: discovery and mode of action; BIOS Scientific Publishers Limited: Oxford, 1995, pp 69-75). On the other hand, PEGylated liposomes are more efficacious than AmBisome, presumably due to prolonged circulation time (van Etten, E. et al. (1995) Antimicrob. Agents Chemother. 39(9),1954). Due to high thermodynamic and kinetic stability in combination with a high surface density of PEG chains, AmB encapsulated by PEG-b-p(HASA), 91% micelles may be long-circulating and released gradually over time.

AmB was released from the micelles in vivo (bioavailable) by or around 6 h based upon time of organism suppression. In vivo time-kill studies suggest that the AmB/PEG-b-p(HASA), 91% formulation is as efficacious as Fungizone, the standard clinical formulation of AmB, against C. albicans in a neutropenic murine model of systemic fungal infection. The polymeric micelle formulation of the present invention has the advantage of reconstitutability after storage in the solid state. Consequently, long-term solution phase stability is not necessary. PEG-b-p(HASA), 91% micelles possess the stability to withstand in vivo dilution and are capable of sustained release of AmB.

It is possible to formulate AmB via a polymeric micelle system based on PEG-b-p(L-Asp) derivatives. Under the experimental conditions, PEG-b-p(HASA) assembles into kinetically stable supramolecular structures capable of encapsulating AmB. Encapsulation of AmB by PEG-b-p(HASA) micelles alters the self-association behavior of AmB compared to free drug, likely due to interactions or complexation between the stearate side chains and AmB Through manipulation of initial loading conditions and altering the extent of stearate esterification, it is possible to exercise some measure of control over the aggregation state of AmB. As the self-aggregation state of the drug has proven to be a good indicator of toxicity and hemolytic activity, encapsulation of AmB in PEG-b-p(HASA) micelles with high levels of stearate attachment might be capable of reducing the toxicity of AmB toward mammalian cells. The ability to alter the equilibrium between the various aggregates might prove quite powerful in terms of drug delivery.

Interactions between highly substituted PEG-b-p(HASA), as indicated by absorption spectroscopy, may result in sustained release of relatively nonself-aggregated AmB, hence the delayed onset of hemolysis. AmB was released from the micelles in vivo (bioavailable) by or around 6 h based upon time of organism suppression. In vivo time-kill studies suggest that the AmB/PEG-b-p(HASA), 91% formulation is as efficacious as Fungizone, the standard clinical formulation of AmB, against C. albicans in a neutropenic murine model of systemic fungal infection. Our polymeric micelle formulation has the advantage of reconstitutability after storage in the solid state. Consequently, long-term solution phase stability is not necessary. PEG-b-p(HASA), 91% micelles are sufficiently stable to withstand in vivo dilution and are capable of sustained release of AmB.

Paclitaxel-loaded PEG-b-PCL micelles were prepared in the same way as described for fenofibrate-loaded micelles. The same proportions of ABC and drug and the same solvent were used. Table 9 shows the results. Similar properties are observed for the paclitaxel-ABC micelles as for the fenofibrate-loaded ABC micelles; thus, the methods of the present invention are applicable to various hydrophobic passenger compounds, those of pharmaceutical importance as well as compounds useful for other purposes.

The encapsulated therapeutic compound-containing compositions, especially those comprising AmB, fenofibrate, paclitaxel, other cancer chemotherapeutics, antibiotics and others, of the present invention are improved with respect to toxicity, stability in aqueous media and with respect to release properties. It has been demonstrated that the present compositions are effective in inhibiting the growth of representative fungal pathogens in vitro. These compositions are similarly effective in vivo after administration by a parenteral route, desirably by intravenous injection, and especially by intravenous perfusion. Other administration in a patient can be by intradermal, intratumoral, intramuscular, intraperitoneal, mucosal and others known to the art. Oral formulations can also be produced using the micelle preparation methods of the present invention. Pathogenic fungi against which the AmB of the present invention are effective include, without limitation, species of Histoplasma, Cryptococcus, Candida, Aspergillus, Blastomycosis, Mucor, Torulopsis, Rhizopus, Absidia, and causative agents of coccidiodomycosis and paracoccidioidomycosis, among others. Anticancer agents such as paclitaxel (Taxol) and the antineoplastic derivatives of paclitaxel are also reduced in toxicity when encapsulated in micelles according to the present invention and delivered by parenteral administration, for example by intravenous injection, intratumoral injection or infusion. It is preferred that the drug-loaded micelles of the present invention are freeze-dried after preparation and stored in the dry state in a manner consistent with maintenance of the activity of the drug, as known in the art for a particular drug. The dry micelles are reconstituted in a pharmaceutically acceptable carrier such as sterile physiological saline or a sterile dextrose solution, e.g., 5% dextrose, and after thorough hydration, they can be filter sterilized (optionally through a 0.22 μm filter) prior to administration. Alternatively, the antibiotic-containing micelles can be formulated in a manner for topical application, using any of a number of art-known technologies for same. The polymeric micelles of the present invention are administered at a similar dosage as is Amphotericin B in prior art liposomal forms.

All references cited in the present application are incorporated by reference herein to the extent that there is no inconsistency with the present disclosure.

The following examples are provided for illustrative purposes, and are not intended to limit the scope of the invention as claimed herein. Any variations in the exemplified articles which occur to the skilled artisan are intended to fall within the scope of the present invention.

### EXAMPLES

### Example 1

Preparation of Methoxy Poly(Ethylene Glycol)-Block-Poly(6-Hydroxyhexyl-L-Aspartamide)-Acyl Conjugates, PEG-b-p(HAZA)

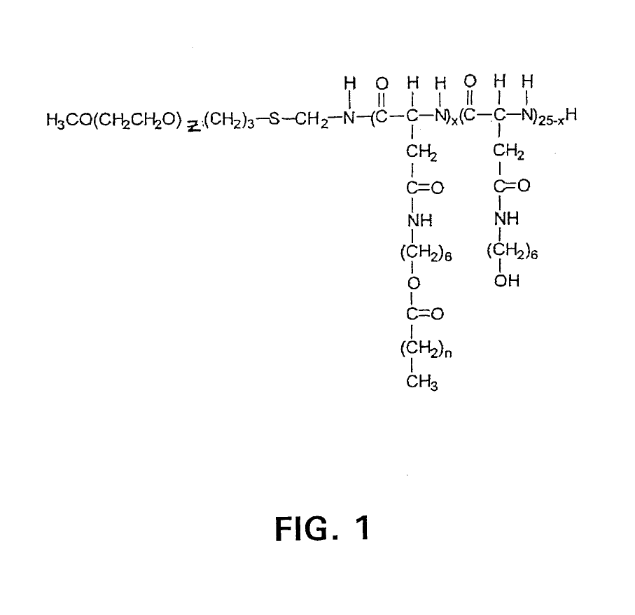

For simplicity, the hydrophilic blocks are referred to as PEG although the blocks are actually methoxy poly(ethylene glycol), mPEG or PEG. The preparation of methoxypoly(ethylene glycol)-block-poly(6-hydroxyhexyl-L-aspartamide), PEG-b-p(6-HHA) and methoxy poly(ethylene glycol)-block-poly(N-hexyl-L-aspartamide)-Z-acid conjugates, PEG-b-p(HAZA) was described (Adams, M. L. et al. (2002) J. Biomat. Sci., Polym. Ed. 13, 991). Briefly, methoxypoly(ethylene glycol)-block-poly(β-benzyl-L-aspartate), PEG-b-PBLA, 12:25 (Nanocarrier, Kashiwashi Ciba, J P), containing a PEG molecular weight of 12,000 g/mol and an average of 25 Asp repeat units was reacted with 6-amino-1-hexanol in freshly distilled dimethylformamide (DMF) in the presence of 2-hydroxypyridine to prepare PEG-b-p(6-HHA). This product was esterified at ambient temperature with excess stearic, lauric, or hexanoic acid in dried CH₂Cl₂ in the presence of dicyclohexylcarbodiimide and dimethylaminopyridine (DMAP). Acetylation of PEG-b-p(6-HHA) was accomplished with excess acetic anhydride in the presence of DMAP. A general chemical structure for the final block copolymer products is given in FIG. 1. Relative acyl attachment was quantified from the ¹H NMR spectra of the conjugates by comparing the relative intensity of the PEG peak at 3.6 ppm to the terminal methyl peak of the acyl chains at 0.9 ppm for the fatty acid conjugates or 2.0 ppm for the acetate methyl (Varian, 500 MHz, Palo Alto, Calif.). On average, the acyl conjugates were prepared with 85% yield. The average degree of substitution for each conjugate is given in Table 1.

In certain experiments, polymer was prepared as follows. The preparation of poly(ethylene glycol)-block-poly(6-hydroxylhexyl-L-aspartamide), PEG-b-p(6-HHA), and poly(ethylene glycol)-block-poly(N-hexyl-L-aspartamide)-stearic acid ester, PEG-b-p(HASA), was described above and in Adams, M. L.; Kwon, G. S. (2002) J. Biomater. Sci., Polym. Ed. 13(9), 991. Briefly, methoxypoly(ethylene glycol)-block-poly(D-benzyl-L-aspartate), PEG-b-PBLA, containing a 12,000 g/mol PEG block and an average of 25 L-Asp repeat units was reacted for 24 h at ambient temperature with excess 6-AH in DMF in the presence of 2-HP. PEG-b-p(6-HHA) was precipitated in cold isopropanol and collected by centrifugation. The pellet was washed with cold isopropanol, followed by chilled ether. The product was dissolved in distilled water and freeze-dried (Labconco, St. Louis, Mo.). Approximately 5 mg of sample was dissolved in 650 μL of DMSO-d₆ and loaded into a 5 mm 535-pp Wilmad NMR tube (Buena, N.J.). Spectra were acquired on a Varian 500 MHz spectrometer (Palo Alto, Calif.) equipped with a 5 mm broad-band probe tuned for proton acquisition and referenced to the solvent multiplet at 2.5 ppm. The loss of the aromatic protons attributed to the phenyl group was observed (spectra not shown). To determine the percent substitution, the methylene resonances centered at 1.25 and 1.40 ppm were integrated relative to the PEG polyether methylene peak at 3.6 ppm. The percent yield for the aminolysis product was 94%.

PEG-b-p(6-HHA) was reacted with either 0.5 or 5 eq. of stearic acid in CH₂Cl₂ in the presence of excess DCC and DMAP at ambient temperature for 5 days. The products were collected as described above. Approximately 5 mg of each conjugate was dissolved in 650 μL of CDCl₃. ¹H NMR spectra were acquired as described above and referenced to the solvent singlet at 7.24 ppm. Comparisons between the relative intensity of the PEG peak and the terminal methyl peak of the stearate chains at 0.9 ppm (spectra not shown) allowed determination of the substitution levels. The final products were prepared in 90% yield.

### Example 2

Drug Loading

80 mg of PEG-b-p(6-HHA) or 5 mg of PEG-b-p(HAZA) was dissolved in 2.0 mL of methanol (MeOH) containing 0.3125 mg/mL of AmB (Chem-Impex, Wood Dale, Ill.). Distilled water (d.H₂O) was added dropwise to the stirring solution at a rate of 0.075-0.090 mL/min (1 drop/10-12 s) to obtain a 50:50 MeOH:d.H₂O mixture. An additional 1 mL of d.H₂O was added to the stirring solution. All samples were sonicated as necessary to obtain clear solutions. Trehalose dihydrate (0.75 g) was then dissolved in the 40:60 MeOH:d.H₂O polymer mixture. In the case of PEG-b-p(6-HHA), 0.6 g of trehalose dihydrate was added instead. The volume was reduced to approximately 1.5 mL via rotary evaporation. The aqueous solution was collected and diluted to a final volume of 5.0 mL with d.H₂O for all polymers except PEG-b-p(6-HHA), which was diluted to 4.0 mL. An additional 0.25 g of trehalose dihydrate was added to the acyl ester solutions and dissolved with the aid of slight stirring. In the case of PEG-b-p(6-HHA), 0.2 g of trehalose dihydrate was added. The aqueous polymer solutions contained 18% trehalose and were sterile-filtered. Aliquots (0.5 mL) were placed in 10 mL serum vials, dipped in N₂ (l) until frozen, and then freeze-dried (Freeze Dry System/Freezone 4.6, Labconco Corporation, Kansas City, Mo.). The freeze-dried samples were stored at 5° C. until use. “Blank” micelles containing only block copolymer were also prepared as described above except that the polymers were initially dissolved in MeOH containing no AmB.

In other experiments, AmB encapsulation was carried out as follows. To determine the effects of stearate esterification on drug-loading, 80 mg of PEG-b-p(6-HHA) or 5 mg of PEG-b-p(HASA), 46 or 91% was dissolved in 2.0 mL of methanol containing 625 μg of AmB. Distilled water (d.H₂O) was added dropwise to the stirring solutions at a rate of 0.075-0.090 mL/min (1 drop/10-12 s) to obtain a 50:50 MeOH:H20 mixture. An additional 1.0 mL of d.H₂O was added to the stirring solutions, followed by 0.75 g of trehalose dihydrate. In the case of PEG-b-p(6-HHA), only 0.6 g of trehalose dihydrate was added. The lyoprotectant was dissolved before MeOH was removed, and the volume was reduced to approximately 1.5 mL via rotary evaporation. The aqueous solutions were collected and diluted to a final volume of 5.0 mL with d.H₂O for all polymers except PEG-b-p(6-HHA), which was diluted to 4.0 mL. An additional 0.25 g of trehalose dihydrate was added to the aqueous PEG-b-p(HASA) solutions and dissolved by stirring. In the case of PEG-b-p(6-HHA), only 0.2 g of trehalose dihydrate was added. The aqueous polymer solutions contained 18% trehalose and were sterile-filtered (0.22 um, ISC BioExpress; Kaysville, Utah). Aliquots (0.5 mL) were immersed in N₂ (l) until frozen, and freeze-dried. To assess the effects of loading conditions on the encapsulation and relative aggregation state of AmB, AmB-loaded PEG-b-p(HASA), 91% micelles were prepared as described above in the presence of 625 or 1250 μg of AmB at different solvent volumes (Table 2). Freeze-dried samples containing AmB were shielded from light. “Blank” micelles, in which AmB was omitted, were prepared as described above. All samples were stored over desiccant at 5° C. until use.

### Example 3

Determination of Aggregation State and AmB Loading

The freeze-dried formulations were reconstituted in 1.0 mL d.H₂O. In order to quantify AmB content, the reconstituted solutions were diluted two-fold with DMF, then diluted appropriately into the linear range with 50:50 DMF:ddH₂O. AmB concentration was quantified via absorbance of monomeric AmB at 412-413 nm (Amersham Pharmacia Biotech Ultraspec 4000, Piscataway, N.J.). Spectra were acquired from 320.0 to 450.0 nm at a rate of 405 nm/min, path length 1.0 cm, and a scan step of 0.1 nm. In order to assess the relative aggregation state of AmB, formulations were reconstituted in d. H₂O and diluted appropriately. A spectrum of 3 μg/mL AmB in PBS (0.0375% DMSO) was also acquired using a 1.0 mm cell (data not shown). All spectra were acquired as described above. In spectra with the final absorbance band centered around 409 nm, the ratio of the first to last peak in the absorbance spectrum served as an indicator of aggregation state. Spectra of 5 μg/mL AmB in DMF and 10 mg/mL AmB is d.H₂O containing 1% DMF were also acquired.

### Example 4

Determination of Micelle Size by Gel Permeation Chromatography (GPC)

All measurements were taken using an Agilent 1100 series HPLC system equipped with a refractive index detector and Agilent GPC analysis software (Agilent Technologies, Palo Alto, Calif.). Freeze-dried PEG-b-p(HAZA) samples, with or without AmB, were reconstituted to 0.5 mg/mL polymer with phosphate-buffered saline, pH=7.2 (PBS), whereas PEG-b-p(6-HHA) formulations were reconstituted to 10 mg/mL. Formulations were injected (100 μL) in duplicate onto a Shodex SB-806M HQ OHpak size exclusion column equipped with a Shodex OHpak SB-G guard column (Showa Denko, New York, N.Y.). The column was equilibrated with PBS mobile phase and calibrated with dextran standards, M_{w}=1−7×10⁶ g/mol (JM Science, Grand Island, N.Y.). The flow rate and column compartment temperature were set to 0.8 mL/min and 37° C., respectively. The weight-averaged molecular weight (M_{w}), number-averaged molecular weight (Mₙ), and polydispersity (D) were calculated with Agilent GPC analysis software (G2071-AA).

### Example 5

Assessment of Hemolytic Activity of AmB/polymer Formulations.

EDTA-anticoagulated bovine blood was diluted in PBS, pH=7.2. The erythrocytes were separated by centrifugation for 10 min at 3000 rpm. The supernatant was removed and discarded. The packed cell volume (PCV) was washed three more times with PBS. The PCV was diluted appropriately in PBS to obtain suspensions of 8×10⁷ and 1.48×10⁸ cells/mL. Cell counts were preformed using an Improved Neubauer Brightline hemacytometer (depth=0.1 mm) from Hausser Scientific (Horsham, Pa.). The AmB/polymer formulations and polymer blanks were brought to room temperature and reconstituted with 1.0 mL of PBS just prior to use. 1.85 mL of the 8×10⁷ cells/mL suspension was incubated at 37° C. in a shaking water bath (75 rpm) with 150 μL of AmB/polymer formulation, polymer blank containing no AmB, or PBS buffer (control) for 24 h. A solution containing 8 mg/mL of amphotericin B in dimethyl sulfoxide (DMSO) was prepared and diluted with buffer to give 6 μg/mL AmB in PBS containing 0.075% DMSO. 1.0 mL of the 1.48×10⁸ cells/mL suspension was incubated with 1.0 mL of 6 μg/mL AmB in PBS (0.075% DMSO) or PBS at 37° C. in the shaking water bath (75 rpm) for 24 h. In all cases, the final AmB concentration in the incubated samples was approximately 3 μg/mL. The final cell concentration was 7.2×10⁷ erythrocytes/mL. Samples, blanks, and controls were withdrawn in triplicate at 1, 9, 16, and 24 h and centrifuged at 3000 rpm for 10 min. The supernatant was collected, and hemoglobin content was determined by absorbance at 542 nm. The values for total cell lysis were obtained by hypotonic hemolysis. Percent hemolysis is reported by 100(Absₛ−Abs_{b})/(Abs₁−Abs_{b}) where Absₛ is the absorbance of the sample, Abs_{b} is the average absorbance of the buffer, and Abs₁ is the average absorbance of the lysed samples. All values are reported as mean±standard deviation.

An 8 mg/mL AmB in DMSO stock solution was prepared. In order to determine the time-dependent hemolytic activity of free drug as a function of concentration, 11.4, 20.4, and 34.0 μL aliquots of the stock solution were diluted to 25.0 mL with PBS to prepare 3.7, 6.5, and 10.9 μg/mL AmB in PBS solutions containing 0.23, 0.41, and 0.68% DMSO, respectively. 1.0 mL of each AmB in PBS solution was then incubated with 1.0 mL of the 1.48×10⁸ cells/mL suspension at 37° C. in a shaking water bath (75 rpm) for 24 h. The final cell concentration was 7.2×10⁷ erythrocytes/mL. The samples were collected over time, and percent hemolysis was determined as described above.

### Example 6

Efficacy of PEG-b-p(HASA)/AmB.

Efficacy was assessed by monitoring viable Candida cells in the kidneys of a neutropenic mouse infected with Candida albicans, a murine model of disseminated fungal infection as described previously by Andes et al. (Andes, D.; Stamsted, T.; Conklin, R. (2001) Antimicrob. Agents Chemother. 45, 9220). A clinical isolate of Candida albicans (K-1) was grown and quantified on Sabaroud Dextrose Agar (SDA). For 24 h prior to infection, the organism was subcultured at 35° C. on SDA slants. A 10⁶ CFU/mL inoculum was prepared by placing six fungal colonies into 5 mL of sterile, depyrogenated normal (0.9%) saline warmed to 35° C. Six-week-old ICR/Swiss specific-pathogen-free female mice were obtained from Harlan Sprague Dawley (Madison, Wis.). All animal studies were approved by the Animal Research Committee of the William S. Middleton Memorial VA Hospital (Madison, Wis.). The mice were weighed (23-27 g) and given intraperitoneal injections of cyclophosphamide to achieve neutropenia. For the purposes of this study, neutropenia was defined as <100 polymorphonuclear leukocytes per mm³. Each mouse was dosed with 150 mg/kg of cyclophosphamide four days prior to infection and 100 mg/kg one day before infection. Disseminated candidiasis was induced via tail vein injection of 100 μL of inoculum, prepared as described above.

The AmB/polymeric micelle formulations or micelle blanks were reconstituted with 1.0 mL of 5% dextrose. Members of the treatment group were each given single 200 μL intravenous (iv) injections of reconstituted AmB/PEG-b-p(HASA), 91% two hours post-infection. Doses were calculated in terms of mg AmB/kg of body weight. Control animals were each given a placebo of “blank” polymeric micelles. At time intervals, twoanimals per experimental condition were sacrificed by CO₂ asphyxiation. The kidneys from each animal were removed and homogenized. The homogenate was diluted 10-fold with 9% saline and plated on SDA. The plates were then incubated for 24 h at 35° C. and inspected for CFU determination. The lower limit of detection for this technique is 100 CFU/mL. To compare the antifungal activity of the AmB/micelle formulations with that of Fungizone, animals were dosed with equivalent doses of AmB as Fungizone as described above. The control animals for the Fungizone group received 200 μL iv injections of 5% dextrose. All results are expressed as the mean CFU per kidney for two animals (four kidneys total). The change in the area under the time-kill curves was calculated by ΔAUC_{TK}=AUC_{control}−AUCₜᵣₑₐₜₘₑₙₜ. Outcomes were compared using ANOVA on ranks.

### Example 7

Fenofibrate Encapsulation in PEG-b-PCL Micelles

Fenofibrate was encapsulated in PEG-b-PCL using the cosolvent evaporation method of the present invention. To prepare the formulation, PEG-b-PCL and drug, with drug w/w % from 0 to 30, were dissolved in acetonitrile with stirring. Water was added gradually (drop-wise) to the block copolymer and drug in acetonitrile while being stirred until 30% water content was reached. Additional water was then added to provide a water content of 80%. The solution remained clear at all steps prior to solvent removal. A final polymeric micelle concentration of 0.5-2.0 mg/mL in water was achieved by removal of the azeotrope and excess water.

After solvent removal, the aqueous micelle samples were filtered (pore size 0.2 Jim) to remove precipitate. Drug content was measured with HPLC at 288 nm before and after filtration to determine encapsulation efficiency. Fenofibrate content in the PEG-b-PCL micelles is measured at 288 nm by HPLC (oactadecyl silane column, 5 cm length, 3 μm diameter; mobile phase, acetonitrile:water, 1:1) after filtration, and loading yield is calculated by the following equation:

$$Percent Yield = \frac{μ g of fenofibrate remaining with the micelles after filtration}{initial μg of fenofibrate before micelle assembly} * 100$$

When micelles are prepared using the assembly methods of the present invention, there is an enhancement in the fenofibrate loading capacity over the equilibrium solubility experiments (See, e.g., FIG. 10).

The equilibrium solubility experiments demonstrate that the larger core forming blocks (4000 and 2500) enhance fenofibrate solubility to roughly 25 μg/mL with 1.0 mg/mL polymer used. However, the cosolvent evaporation achieves levels over 8 times that amount. With cosolvent evaporation, the (5000: 1000) composition lost drug during the loading and had a lower loading efficiency, however it is higher than that of the solubility experiments. Both of the higher core-forming blocks had a high efficiency of loading. The PEG-b-PCL (5000:4000) had nearly 100% yields at all amounts loaded except for 30% w/w. Yet, at 30% w/w fenofibrate, the amount of fenofibrate encapsulated still increased. A 253 μg/mL was achieved as the maximum drug loaded using the novel assembly conditions. In the other compositions, (5000:1000) and (5000:2500), an optimization of loading was reached. At 10% w/w fenofibrate, PEG-b-PCL (5000: 1000) reached a maximum level of fenofibrate encapsulation of approximately 75 μg/mL. The (5000:2500) mirrored the larger core block forming in terms of encapsulation capacity until 25% w/w fenofibrate was achieved. At 25% w/w fenofibrate, the (5000:2500) micelle formulation only retained 77% of the total fenofibrate loaded while the (5000:4000) retained 92%. At 30% w/w fenofibrate the (5000:4000) decreased to 76% loading and the (5000:2500) composition had a 55% yield. It is important to note that (5000:4000) and (5000:2500) micelles exhibit an appearance of a secondary population with increasing drug content.

In addition, the size and polydispersity of the drug-loaded micelles were determined with Dynamic Light Scattering (DLS). DLS measurements were performed at 25° C. and at angles ranging from 45° to 160°. PEG-b-PCL (5:4) was dissolved in acetonitrile and water was added slowly to achieve the desired water to acetonitrile ratio and polymer concentration. The samples were then allowed to equilibrate overnight with agitation. A dust-free analysis was ensured, by washing the 0.45 μm filter with the appropriate cosolvent mixture two times prior to filtering the samples. A total of 40 scans over a 10 minute period was collected for each sample. The data were analyzed using the QelsmonNT software. From the knowledge of solvent viscosity (η) and after correcting for the change solvent refractive index caused by the use of cosolvent mixtures, the radius of hydration can be determined using the Einstein relationship:

$${R}_{h} = \frac{{k}_{g} T}{6 πη D}$$

$${k}_{g} \text{:} Boltzmann constant$$

$$T \text{:} absolute temperature$$

$$η \text{:} viscosity of the solvent$$

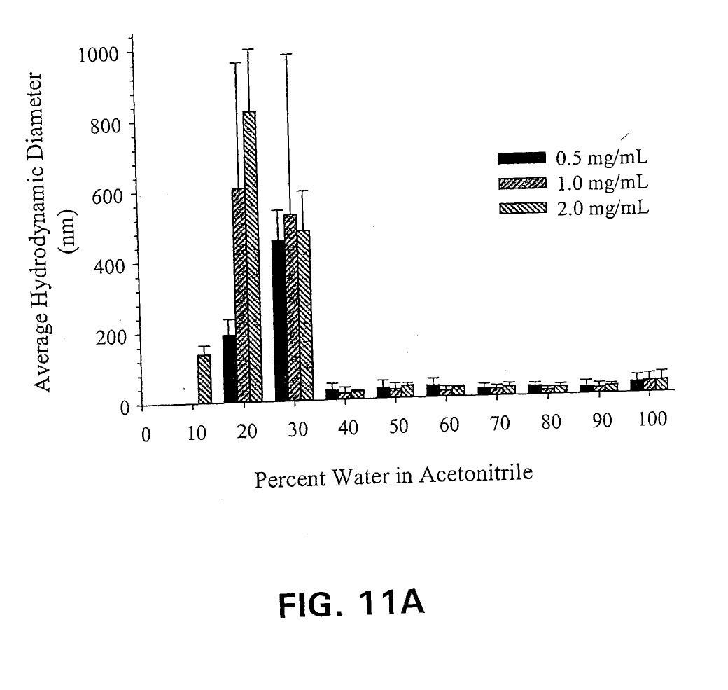

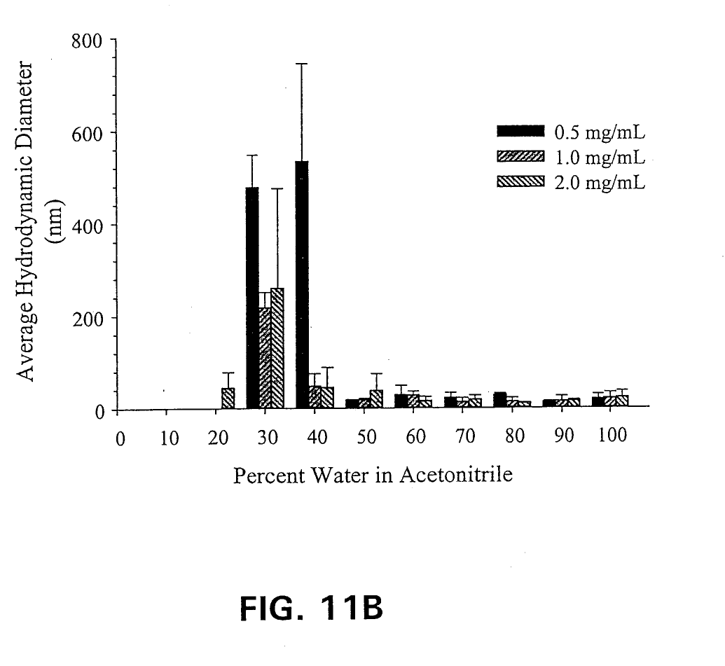

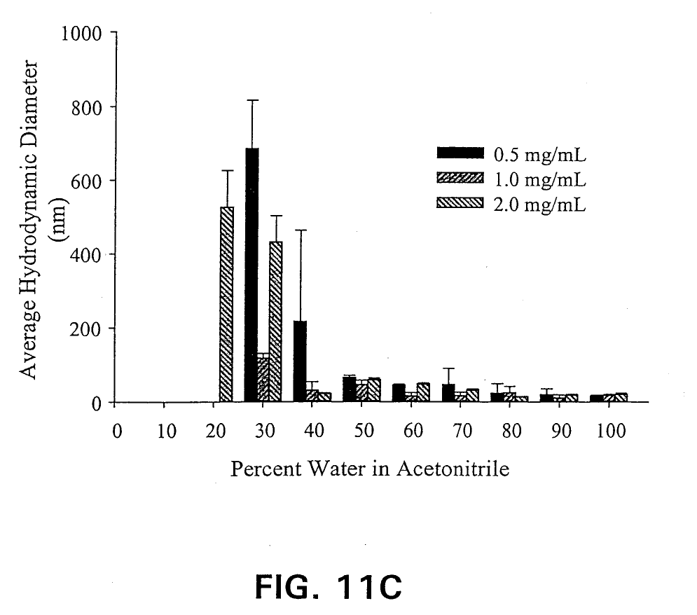

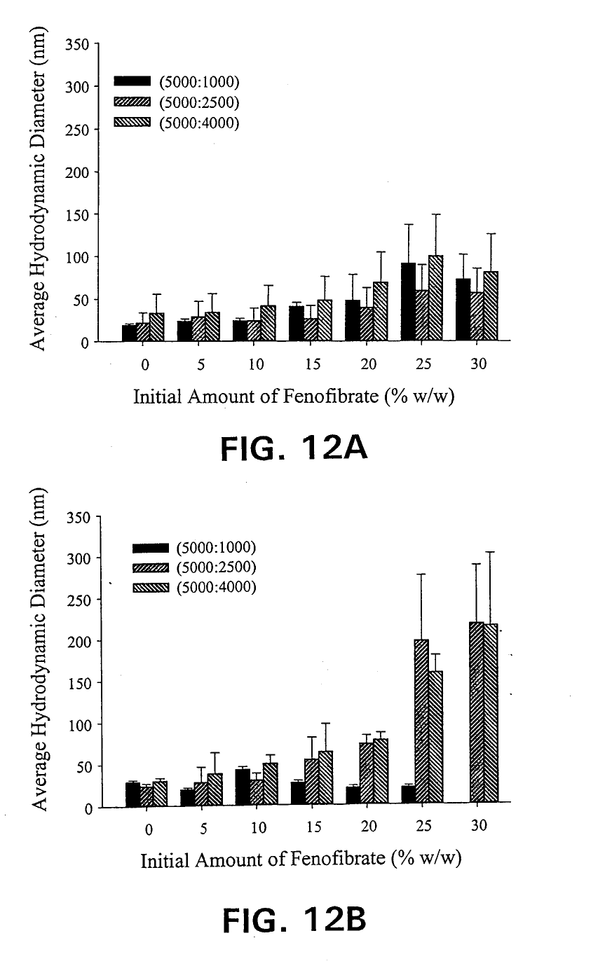

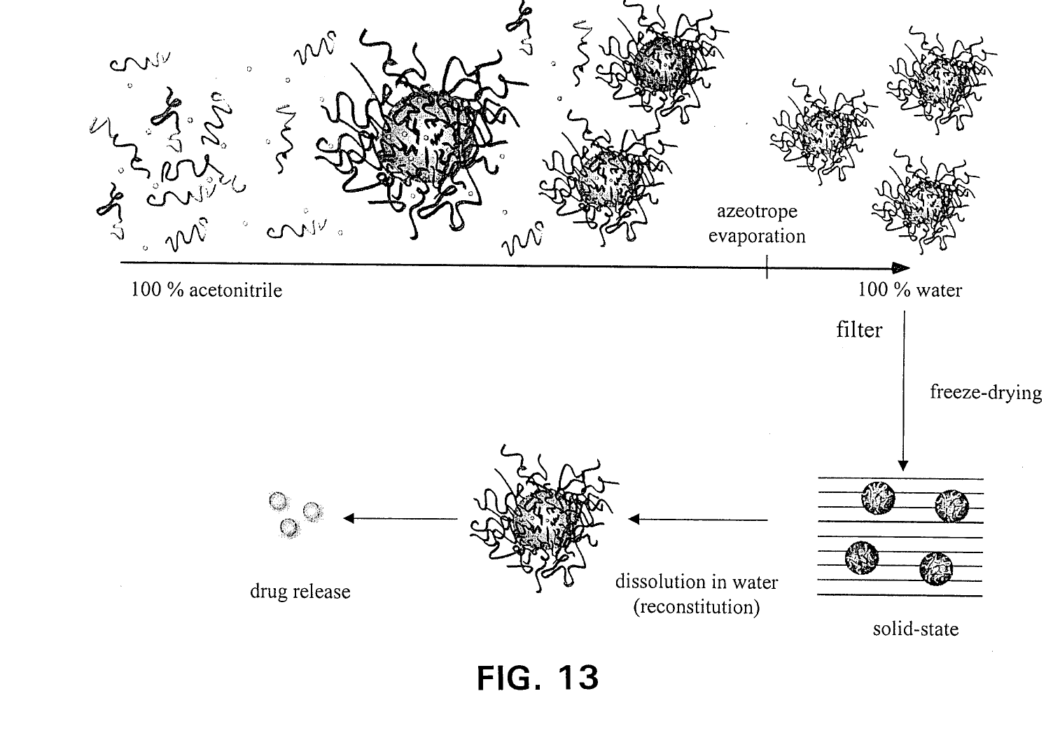

Next, the Einstein relationship was used to calculate the radius of hydration of the particles as water was added drop-wise to the polymer solutions. The average radius vs. water content data are shown in FIGS. 11A, 11B, 11C for PEG-b-PCL 5000:4000, 5000:25000, 5000:1000, respectively). The initial hydrodynamic radius of hydration of PEG-b-PCL (5000:4000) at 0.1% w/w was 2.65 nm. This value is significantly smaller than the radius of a polymeric micelle, which is expected to be 20-60 nm. Therefore, DLS experiments confirm that in ACN the polymer strands are not aggregated. The cwc of this concentration of polymer is approximately 10% water and there is a peak at about 30% water content. However, above 50% water, the size remains constant because the data in FIGS. 11A-11C have been normalized to the cosolvent refractive index and viscosity.

Between 20 and 60% water content, two distinct populations of particles were observed. At 20% water content, 20% of the sample had a radius of 238 nm while 80% of the sample was 50 nm. Further addition of water to 30% resulted in a small population of 30% having a size of 20 nm and the rest at near 200 nm. By 40% water content, the populations were equal and were 200 and 16 nm. With 50% water content, the smaller sized population of 15 nm in diameter was more prevalent (60%), and the other population diminished to 40 nm. From a thermodynamic perspective, the presence of two different populations of micelles is difficult to explain, and therefore, without wishing to be bound by any particular theory, it is postulated that at water contents ranging from 20 to 50%, the polymer micelles have swollen elongated shapes. Because the shape is elongated, the hydrodynamic radius of the length is different from the width, and the sample appears to have two populations of structure. However, at water contents greater than 50%, the micelles are more spherical, and a uni-modal distribution is observed. The hydrodynamic radius of the micelles was found to be 18-20 nm. These values are appropriate for diblock copolymer micelles to be used to incorporate hydrophobic passenger molecules such as therapeutic agents.

In the formation of polymeric micelles, it is expected that the polymer chain rearrangement is slow. Therefore it was necessary to determine whether the formation of spherical structures is a rate-limiting step during the preparation of the micellar solution. It has been shown in the literature that water added drop-wise through the cwc results in small, spherical, and unimodal distributions of micelles (Lavasanifar et al. (2001) supra). In the present study we determined the point where it was no longer necessary to add water drop-wise and still achieve small and spherical micellar structures. Therefore, adding water drop-wise through the cwc followed by rapid addition of water was investigated.

To each sample of PEG-b-PCL (5000:4000), water was added drop-wise to reach 20, 40, 60 and 80% water contents and sizes of the polymer micelles were measured with DLS. Then instead of continuing to add water drop-wise, samples at 20, 40, and 60% water were quenched by adding water directly to 80%. The results indicated that it was not crucial to add water gradually after cwc.

In summary, through the addition of water after the cwc, the micelles assemble into swollen structures 200-1000 nm, and with the further addition of water, the cores collapse, forming relatively monodispersed micelles of 20-60 nm in diameter.

PEG-b-PCL micelles were filtered before measurement with a 0.22 gm nylon filter. At loading levels below 15% w/w, the PEG-b-PCL micelles were close to the size without drug loading. The PEG-b-PCL (5000: 1000) diameter with no drug present was 20 nm. As drug content was increased to 5 and 10% w/w, the sizes were both 24 nm. After 15% w/w drug loading, the sizes of the PEG-b-PCL micelles were 40 nm and increased to 48 nm at 20% w/w. The high levels of drug loading (25 and 30% w/w) with PEG-b-PCL (5000: 1000) the hydrodynamic diameter reached 92 and 72 nm respectively. It is interesting to note that with the PEG-b-PCL (5000:1000) micelles, the diameters increased with drug loading, although drug content after filtration was similar to 5% w/w samples.

PEG-b-PCL (5000:2500) micelle diameter also increased as drug loading increased. The hydrodynamic diameter of the micelles was 40 nm at higher loading levels as opposed to 20-30 nm with lower drug levels (up to about 20% w/w). The size of the PEG-b-PCL micelles increased, at levels of 25 and 30% w/w, to 59 and 59 nm, respectively. The largest core forming block of 4000 had the largest recorded diameters overall. After 5% w/w fenofibrate loading, the size of the PEG-b-PCL micelles increased gradually 41 nm at 10% w/w and 48 nm at 15% w/w. At the drug loading levels of 20% w/w fenofibrate, the size was 69 nm and continued to increase to 100 nm at 25% w/w. With the highest level of drug loading of 30% w/w, the hydrodynamic diameter was 81 nm. The results described herein demonstrate that it is possible to encapsulate fenofibrate into PEG-b-PCL micelles effectively and to enhance the aqueous solubility of the drug.

### Example 8

Materials

Dichloromethane (CH₂Cl₂) and D(+) trehalose dihydrate were purchased from Sigma (St. Louis, Mo.). Deuterated chloroform (CDCl₃), deuterated dimethyl sulfoxide (DMSO-d₆), N,N-dimethylformamide (DMF), dimethylaminopyridine (DMAP), 6-amino-1-hexanol (6-AH), 2-hydroxypyridine (2-HP), stearic acid, and molecular sieves (4A) were purchased from Aldrich (Milwaukee, Wis.). CH₂Cl₂ was dried with molecular sieves, and DMF was distilled under vacuum prior to use. Methanol (MeOH) and dimethyl sulfoxide (DMSO) was purchased from Fisher Scientific (Fair Lawn, N.J.). Dicyclohexylcarbodiimide (DCC) was purchased from Lancaster (Windham, N.H.). Amphotericin B (AmB) was obtained from Chem-Impex (Wood Dale, Ill.). Methoxypoly(ethylene glycol)-block-poly(β-benzyl-L-aspartate) was provided by NanoCarrier (Kashiwa, Japan). PEG-block-poly(hydroxy-alkyl L-aspartamide block copolymers were obtained from K. Kataoka; they are described in U.S. Pat. No. 5,449,513; see also a description of the synthesis of PEG-b-PBLA block copolymers in Yokoyama et al. (1992) Bioconj. Chem. 3, 295. PEG-b-PCL diblock copolymers were purchased from Polymer Source Inc., Dorval, Quebec, Canada. All chemicals were reagent grade or above, and used without additional purification unless stated otherwise.

A clinical bloodstream isolate of Candida albicans (K-1) was used for in vivo efficacy experiments (Andes et al., 2000, supra). Sabouraud dextrose agar (SDA) slants were from Difco Laboratories (Detroit, Mich.). Fungizone was obtained from Bristol-Myers Squibb (Princeton, N.J.) and stored at −70° C. until use.

TABLE 1
PEG-b-PBLA derivatives used for the encapsulation of amphotericin B.

| | Acyl chain length | | | |
|---|---|---|---|---|
| Polymer | (#C) | n (CH₂) | x | Substitution Level (%) |
| PEG-b-p(6- | 0 | n/a | 24.8 | 99.3 ± 0.1 |
| HHA) | | | | |
| PEG-b- | 2 | 0 | 23.7 | 94.6 ± 0.6 |
| p(HAAA) | | | | |
| PEG-b- | 6 | 4 | 21.9 | 87.4 ± 0.5 |
| p(HAHA) | | | | |
| PEG-b- | 12 | 10 | 21.4 | 85.6 ± 0.5 |
| p(HALA) | | | | |
| PEG-b- | 18 | 16 | 22.7 | 90.7 ± 0.9 |
| p(HASA) | | | | |

abbreviations and structures (n and x refer to FIG. 1).

TABLE 2
Results of AmB Encapsulation in PEG-b-pHAZA Micelles.

| | [polymer], | | AmB:polymer | Encapsulation |
|---|---|---|---|---|
| Polymer | mg/mL | [AmB], μM* | (mol:mol) | Efficiency |
| PEG-b-p(6- | 10 | 44.9 ± 0.8 | 0.08 ± 0.01 | 0.66 ± 0.01 |
| HHA) | | | | |
| PEG-b- | 0.5 | 53.2 ± 2.1 | 1.94 ± 0.08 | 0.79 ± 0.03 |
| p(HAAA) | | | | |
| PEG-b- | 0.5 | 36.9 ± 0.8 | 1.43 ± 0.03 | 0.55 ± 0.01 |
| p(HAHA) | | | | |
| PEG-b- | 0.5 | 53.2 ± 0.8 | 2.25 ± 0.03 | 0.70 ± 0.03 |
| p(HALA) | | | | |
| PEG-b- | 0.5 | 42.7 ± 0.3 | 1.96 ± 0.01 | 0.63 ± 0.01 |
| p(HASA) | | | | |

*designates the level of AmB upon reconstitution with 1 mL of d.H₂O.

TABLE 3
Block Copolymers with Varying Degrees of Substitution.

| | | Degree | |
|---|---|---|---|
| | Stearic Acid Reacted, | of Substitution, | |
| Polymer | eq. acid/eq. OH | avg. ± std. dev^{b}. | x^{c} |
| PEG-b-p(6-HHA) | n/a^{a} | 99.3 ± 0.5 | 25 |
| PEG-b-p(HASA), 46% | 0.5 | 46.3 ± 1.0 | 12 |
| PEG-b-p(HASA), 91% | 5 | 90.7 ± 0.9 | 23 |

^{a}Not applicable.
^{b}Determined by ¹H NMR (Varian, 500 MHz) from five integrations per polymer batch.
^{c}Refers to FIG. 1 and represents the average number of conjugated groups per 25 L-Asp repeat units.

TABLE 4
GPC results for polymeric micelle samples with and without AmB.

| Esterification | AmB:polymer, | M_{w} × 10⁶, | Mₙ × 10⁶ | |
|---|---|---|---|---|
| Level, % | mol:mol | g/mol | g/mol | Polydispersity |
| 0 | 0.0 | n/a | n/a | n/a |
| 0 | 0.1 | n/a | n/a | n/a |
| 46 | 0.0 | 3.9 | 1.6 | 2.4 |
| 46 | 1.9 | 2.5 | 1.5 | 1.7 |
| 91 | 0.0 | 5.0 | 2.4 | 2.1 |
| 91 | 1.2 | 4.9 | 2.1 | 2.3 |
| 91 | 2.0 | 4.7 | 2.0 | 2.3 |
| 91 | 3.6 | 2.9 | 1.8 | 1.6 |

TABLE 5
Effect of loading conditions on AmB encapsulation by
PEG-b-p(HASA), 91%.

| Initial | MeOH | [AmB] | AmB:PEG-b- | |
|---|---|---|---|---|
| Level of | Volume, | loaded, | p(HASA), | Encapsulation |
| AmB, μg | mL | μg/mL* | 91%, mol:mol | Efficiency |
| 625 | 4.0 | 26.4 ± 1.0 | 1.2 ± 0.1 | 0.39 ± 0.01 |
| 625 | 2.0 | 42.7 ± 0.3 | 2.0 ± 0.1 | 0.63 ± 0.01 |
| 1250 | 4.0 | 78.5 ± 2.8 | 3.6 ± 0.1 | 0.58 ± 0.01 |

*Upon reconstitution with 1.0 mL of d.H₂O.

TABLE 6
Lyoprotectant Effect on Reconstituted PEG-b-PCL (5000:4000) Micelles*

| | Sugar/PEG-b- | Hydrodynamic | |
|---|---|---|---|
| | PCL | diameter | Standard Dev. |
| | (w/w) | (nm) | n + 3 |
| Sucrose | 5 | 52.6 | 10.5 |
| | 10 | 23.9 | 9.5 |
| | 25 | 24.0 | 10.0 |
| | 50 | 26.5 | 14.9 |
| | 125 | 21.9 | 6.4 |
| Trehalose 2H₂O | 5 | 460.3 | 203.4 |
| | 10 | 458.0 | 204.0 |
| | 25 | 58.9 | 42.6 |
| | 50 | 24.0 | 158.7 |
| | 125 | 22.0 | 25.0 |
| Raffinose 5H₂O | 5 | 575.7 | 165.1 |
| | 10 | 738.4 | 213.4 |
| | 25 | 48.9 | 254.2 |
| | 50 | 78.2 | 43.3 |
| | 125 | 39.2 | 2.7 |

*Reconstituted in water to achieve 2.0 mg/ml PEG-b-PCL

TABLE 7
Drug Loading and Stability Summary

| | | | |
|---|---|---|---|
| PEG-b-PCL | (5000:1000) | (5000:2500) | (5000:4000) |

Without Fenofibrate

| | | | |
|---|---|---|---|
| DLS | $\frac{2 {R}_{h} ( nm )}{19.0 ( + / - ) 2.1}$ | $\frac{2 {R}_{h} ( nm )}{21.5 ( + / - ) 12.2}$ | $\frac{2 {R}_{h} ( nm )}{33.4 ( + / - ) 22.1}$ |
| GPC | $\frac{{M}_{w} ( {10}^{6} g / mol}{detection limited}$ | $\frac{{M}_{w} ( {10}^{6} g / mol )}{1.50 ( + / - ) 0.12}$ | $\frac{{M}_{w} ( {10}^{6} g / mol}{2.22 ( + / - ) 0.14}$ |

Fenofibrate: initial % w/w = 10

| | | | |
|---|---|---|---|
| Loaded Fenofibrate | $\frac{Percent yield}{28.3 ( + / - ) 8.5}$ | $\frac{Percent yield}{95.1 ( + / - ) 1.8}$ | $\frac{Percent yield}{92.4 ( + / - ) 2.8}$ |
| DLS | $\frac{2 {R}_{h} ( nm )}{24.3 ( + / - ) 2.7}$ | $\frac{2 {R}_{h} ( nm )}{28.3 ( + / - ) 14.7}$ | $\frac{2 {R}_{h} ( nm )}{41.4 ( + / - ) 24.2}$ |
| GPC | $\frac{{M}_{w} ( {10}^{6} g / mol}{2.56 ( + / - ) 0.63}$ | $\frac{{M}_{w} ( {10}^{6} g / mol}{2.22 ( + / - ) 0.29}$ | $\frac{{M}_{w} ( {10}^{6} g / mol}{5.25 ( + / - ) 0.44}$ |

PEG-b-PCL concentration: 1 mg/mL, n = 3

TABLE 8
Comparisons of two known methods to create drug loaded
PEG-b-PCL micelles.
Fenofibrate Drug Loading

| | PEG-b-PCL | | Final Drug/ | |
|---|---|---|---|---|
| | (5000:4000) | Fenofibrate | Polymer | % Yield |
| Method | (mg) | (mg) | Mole Ratio | of Drug |
| Cosolvent | 51 | 7.2 | 3.1 | 88 |
| Evaporation* | | | | |
| Dialysis | 54 | 7.5 | 1.1 | 31 |

Amphotericin B Drug Loading

| | PEG-b-PCL | | Final Drug/ | |
|---|---|---|---|---|
| | (5000:4000) | AmB | Polymer | % Yield |
| Method | (mg) | (μg) | Mole Ratio | of Drug |
| Cosolvent | 20.1 | 470 | 0.11 | 48 |
| Evaporation | | | | |
| Dialysis | 20.2 | 460 | 0.055 | 25 |

Fenofibrate and amphotericin B were encapsulated into PEG-b-PCL (5000:4000) micelles using the dialysis method and the cosolvent evaporation method of the present invention.

TABLE 9
Paclitaxel Loaded Micelles

| | | | | |
|---|---|---|---|---|
| | | Before | | |
| | | Freeze-drying | | After Freeze-drying |
| % | % | DLS | SEC | DLS |
| w/w | yield | 2Rₕ (nm) | M_{w} (g/mol* 10⁶) | 2Rₕ (nm) |

PEG-b-PCL (5000:2500)

| | | | | |
|---|---|---|---|---|
| 0 | 0 | 21.5 (+/−) 12.2 | 1.52 (+/−) 0.12 | 29.1 (+/−) 6.8 |
| 1 | 98.1 | 28.7 (+/−) 4.9 | | 35.0 (+/−) 26.1 |
| 5 | 74.1 | 35.0 (+/−) 24.4 | 1.93 (+/−) 0.091 | 49.6 (+/−) 32.6 |
| 10 | 70.47 | 35.8 (+/−) 22.4 | | 57.8 (+/−) 37.0 |

PEG-b-PCL (5000:4000)

| | | | | |
|---|---|---|---|---|
| 0 | 0 | 33.4 (+/−) 22.1 | 2.22 (+/−) 0.14 | 36.7 (+/−) 27.7 |
| 1 | 87.6 | 48.3 (+/−) 34.4 | | 57.6 (+/−) 39.5 |
| 5 | 85.8 | 49.1 (+/−) 33.4 | 3.11 (+/−) 0.214 | 60.7 (+/−) 42.1 |
| 10 | 15.8 | 74.9 (+/−) 52.9 | | 93.6 (+/−) 68.3 |

What is claimed is:

1. A method for preparing a micelle composition comprising a hydrophobic passenger compound and an amphiphilic block copolymer, said method comprising the steps of:
(a) dissolving the hydrophobic passenger compound and the amphiphilic block copolymer in a solvent which is miscible with water and has a boiling temperature lower than that of water;
(b) adding water at a rate and in an amount sufficient to promote micelle formation;
(c) adding water to excess over that to promote micelle formation;
(d) optionally adding a lyoprotectant;
(e) after step (c) or (d), evaporating to remove solvent with the result of leaving a substantially solvent-free aqueous micelle solution; and
(f) optionally after steps (d) and (e), evaporating the aqueous solution to produce a dried micelle composition.

2. The method of claim 1, wherein the solvent is selected from the group consisting of acetonitrile, methanol, ethanol, and acetone.

3. The method of claim 1, wherein the lyoprotectant is selected from the group consisting of sucrose, trehalose, raffinose and poly(ethylene glycol).

4. The method of claim 1, wherein the hydrophobic passenger compound is a therapeutic agent.

5. The method of claim 1, wherein the amphiphilic block copolymer is selected from the group consisting of a methoxypoly(oxyethylene)-block-poly(ε-caprolactone) and a methoxypoly(oxyethylene) poly(ethylene glycol)-block-poly(N-hexyl-L-aspartamideacyl ester) where the acyl is selected from the group consisting of acetate, hexaoate, laurate and stearate.

6. The method of claim 4, wherein the therapeutic agent is selected from the group consisting of fenofibrate, paclitaxel, adriamycin and amphotericin B.

7. The method of claim 6, wherein the therapeutic agent is amphotericin B and the amphiphilic block copolymer is methoxy poly(ethylene glycol)-block-poly(N-hexyl-L-aspartamide-stearate ester).

8. The method of claim 7, wherein the stearate acyl moiety is present at a level of substitution from 70% to 95%.

9. The method of claim 7, wherein the solvent is acetonitrile and the lyoprotectant is selected from the group consisting of sucrose, trehalose and raffinose.

10. The method of claim 6, wherein the therapeutic agent is fenofibrate and the amphiphilic block copolymer is methoxy poly(ethylene glycol)-block-(ε-polycaprolactone) (CH₃O—(CH₂—CH₂—O—)N—(CO(CH₂)₅—O—)M—CO(CH₂),—OH, where N is from 50 to 150 and M is from 8 to 175.

11. The method of claim 10, wherein N is from 75 to 125 and wherein M is from 8 to 40.

12. The method of claim 11, wherein N is 113 and M is 8, 21 or 34.

13. The method of claim 10, wherein the solvent is acetonitrile and the lyoprotectant is selected from the group consisting of sucrose, trehalose and raffinose.

14. The method of claim 10, wherein the lyoprotectant is added at a ratio of weight of polymer to weight of lyoprotectant from 1:1 to 1:10.

15. A method for reducing the toxicity of a polyene antibiotic, said method comprising the step of incorporating a polyene antibiotic in poly(ethylene glycol)-block-poly(N-hexyl-L-aspartamide)-acyl ester micelles, wherein the general chemical structure for the final block copolymer products is given in FIG. 1, where n can be from 8 to 28, z can be from about 250 to about 300; and wherein 25-x is from 1 to 5, said method comprising the steps of:
(a) dissolving the polyene antibiotic and the poly(ethylene glycol)-block-poly(N-hexyl-L-aspartamide)-acyl ester in a solvent;
(b) adding water in an amount and at a rate sufficient to promote micelle formation;
(c) adding water to excess over that sufficient to promote micelle formation;
(d) removing solvent to produce a substantially solvent-free aqueous solution of micelles;
(e) optionally, after step (c) or step (d), adding and dissolving a lyoprotectant;
(f) optionally after step (e), removing the water to produce a dried micelle preparation.

16. The method of claim 15, wherein the polyene antibiotic is amphotericin B and the poly(ethylene glycol)-block-poly(N-hexyl-L-aspartamide)-acyl ester is a poly(ethylene glycol)-block-poly(N-hexyl-L-aspartamide)-stearate ester.

17. The method of claim 15, wherein the lyoprotectant is selected from the group consisting of sucrose, trehalose, raffinose and poly(ethylene glycol).

18. The method of claim 15, wherein n is 12 to 18.

19. The method of claim 18, wherein n is 16.

20. The method of claim 15, wherein z is 273.

21. The method of claim 15, wherein the polyene is Amphotericin B.

22. The product of the method of claim 15.

23. The product of claim 22 further comprising a pharmaceutically acceptable carrier.

24. The composition of claim 23, wherein the pharmaceutically acceptable carrier is a dextrose solution.
